(19) United States
(12) Patent Application Publication
(10) Pub. No.: US 2011/0171290 A1
(43) Pub. Date: Jul. 14, 2011

(54) INHIBITION OF ANGIOGENESIS AND TUMOR METASTASIS

(75) Inventors: Peter Altevogt, Neckargemund (DE); Yasmin Issa, Mainz (DE); Philipp Beckhove, Heidelberg (DE); Daniel Nummer, Mainz (DE)

(73) Assignee: Deutsches Krebsforschungszentrum Stiftung des offentlichen Rechts

(21) Appl. No.: 12/937,964

(22) PCT Filed: Apr. 16, 2009
(86) PCT No.: PCT/EP09/02792

§ 371 Date: Mar. 29, 2011

Related U.S. Application Data
(60) Provisional application No. 61/071,167, filed on Apr. 16, 2008.

(51) Int. Cl. A61K 39/395; A61K 9/127; A61P 9/00

(52) U.S. Cl. 424/450; 424/172.1; 424/133.1; 424/142.1; 424/141.1

(57) ABSTRACT

The present invention relates to an anti L1-CAM antibody for use in a method for the treatment of a disease in a patient which can be treated by inhibition of angiogenesis, wherein the administration of said anti L1-CAM antibody results in the inhibition of angiogenesis and/or tumor metastasis.

The present invention relates to the treatment of a disease which can be treated by inhibition of angiogenesis as well as to the prevention of tumor metastasis formation.

Angiogenesis is the process by which new blood vessels are formed (Folkman and Klagsbrun, 1987). It is essential for normal body activities such as reproduction, development, and wound repair. Although the entire process of angiogenesis is not completely understood, it is believed that the process involves a complex set of molecules that interact with each other to regulate the growth of endothelial cells, the primary cells of capillary blood vessels. Under normal conditions, these molecules maintain the cells in quiescent state, i.e., a state of no capillary growth, for prolonged periods of time that may last for as long as weeks or, in some cases, decades. However, when necessary, such as during wound healing, these molecules will promote rapid proliferation and turnover of the cells within a five day period (Folkman, 1992).

Endothelial cells are those cells making up the endothelium, the monolayer of simple squamous cells which lines the inner surface of the circulatory system. These cells retain a capacity for cell division, although they proliferate slowly under normal conditions. In normal vessels the proportion of proliferating endothelial cells is especially high at branch points in arteries, where turbulence and wear seem to stimulate turnover. Endothelial cells have also the capacity to migrate, a process important in angiogenesis. Endothelial cells form new capillaries in vivo when there is a need for them, such as during wound repair or when there is a perceived need for them as in tumor formation. The formation of new vessels is termed angiogenesis, and can involve molecules (angiogenic factors) which can be mitogenic or chemoattractant for endothelial cells. During angiogenesis, endothelial cells can migrate out from an existing capillary to begin the formation of a new vessel. In vitro studies have documented both the proliferation and migration of endothelial cells; endothelial cells placed in culture can proliferate and spontaneously develop capillary tubes.

Although angiogenesis is a highly regulated process under normal conditions, many diseases are characterized by persistent unregulated angiogenesis. For example, the growth and metastasis of solid tumors are also dependent on angiogenesis (Folkman et al., 1989; Folkman, 1992). Tumors which enlarge to greater than 2 mm must obtain their own blood supply and do so by inducing the growth of new capillary blood vessels. These new blood vessels embedded within the tumor provide a means for tumor cells to enter the circulation and metastasize to distant sites such as liver, lung, or bone. The role of angiogenesis is not limited to solid tumors, but has also been shown for hematogenous tumors (Perez-Atayde et al., 1997).

Malignant tumors become more and more common and they pose a significant threat to human lives. There are conventional means to treat malignant tumors, such as surgery, chemotherapy and radiotherapy. The type or stage of the cancer can determine which of the three general types of treatment will be used. An aggressive, combined modality treatment plan can also be chosen e.g. surgery can be used to remove the primary tumor and the remaining cells are treated with radiation therapy or chemotherapy. In general, chemotherapeutic agents and radiotherapy are unable to distinguish cancer cells from normal cells. Moreover, these therapies are inefficient for patients suffering from tumors in an advanced stage, therefore people tried to develop new strategies. Although there were great expectations in tumor gene therapy, there has been no clinical breakthrough so far. The use of hormone therapy and immunotherapy remains limited to distinct cases and cancer types. Research to identify more effective drugs for treating advanced disease continues.

Pancreatic cancer is a highly aggressive, treatment refractory disease and the fourth leading cause of cancer death in the U.S. (Nitecki et al., 1995). The median survival time is less than 6 months. Potential curative resection of the tumour is still the only option that offers a chance for cure, but can only be performed in about 10 to 15% of pancreatic cancer patients. The prognosis for patients is still poor and more than 80% die within 5 years after surgery. Due to its exocrine functions primary pancreatic cancer often develop multiple metastases.

In addition to tumor malignancies, a variety of diseases are characterized by persistent unregulated angiogenesis such as ocular diseases (e.g. macular degeneration such as age related macular degeneration, or retinopathy such as proliferative diabetic retinopathy) or chronic inflammatory diseases, especially arthritic diseases (e.g. rheumatoid arthritis or osteoarthritis), inflammatory skin diseases (e.g. dermatitis or psoriasis) neuroinflammatory diseases (e.g. multiple sclerosis), Crohn's disease, or stomach ulcers (Folkman, 1995; Carmeliet, 2005; Adamis et al., 1999; Costa et al., 2007). Further conditions that are considered to be affected by angiogenesis are obesity, asthma, diabetes, cirrhosis, endometriosis, AIDS and bacterial infections.

As the important contribution of angiogenesis to numerous diseases has been recognised, the inhibition of angiogenesis has been proposed as a promising strategy for the treatment of these diseases (Folkman, 1995; Ferrara and Kerbel, 2005). Several agents, most of them targeting the VEGF pathway, have been tested in clinical trials. So far the monoclonal antibody bevacizumab has been approved for the treatment of colon and non-small cell lung cancer, and the small molecules sunitinib and sorafenib have been approved for the treatment of renal cell carcinoma, imatinib-resistant gastrointestinal stromal tumors. In the field of ocular diseases, the antibody fragment ranibizumab and the aptamer drug pegaptanib were approved for the treatment of age related macular degeneration. Hence, it has been proven that the concept of angiogenesis inhibition can successfully be implemented into the treatment of angiogenesis associated diseases. Still there is a constant demand of new antiangiogenic therapies because there is emerging evidence, that patients may become resistant to existing antiangiogenic agents during the course of the treatment (Ferrara and Kerbel, 2005).

L1-CAM is a 200-220 kDa transmembrane glycoprotein of the immunoglobulin (Ig) superfamily composed of six Ig-like domains and five fibronectin type III repeats followed by a transmembrane region and a highly conserved cytoplasmic tail (Moos et al., 1988). L1-CAM was first described in the nervous system, where it is important for cell migration and axon outgrowth (Schachner, 1997). Throughout the present invention, the terms L1, L1-CAM and L1CAM are used interchangeable.

Recently, overexpression of L1-CAM in ovarian and endometrial carcinomas has been reported to correlate with bad prognosis and is associated with metastases formation in melanoma and colorectal carcinoma (Fogel et al., 2003; Thies et al., 2002; Kaifi et al., 2007). L1-CAM expression was also described in other carcinomas such as neuroblastomas and pancreatic adenocarcinoma (Patel et al., 1992; Sebens Müerköster et al. 2007). L1-CAM is weakly expressed by hematopoietic cells and was also noted on certain endothelial cells (EC) (Ebeling et al., 1996; Felding-Habermann et al., 1997). Functionally, L1-CAM can interact with itself (homophilic) but also with a variety of heterophilic ligands such as integrins, CD24, neurocan, neuropilin-1 (NRP-1) and other members of the neural cell adhesion family (Brummendorf et al., 1998). L1-CAM associates with NRP-1 to form a semaphorin3A (Sema3A) receptor complex important for axon guidance responses (Castellani et al., 2002). NRP-1 is a single spanning transmembrane glycoprotein, initially characterized as a neuronal receptor for specific secreted members of the semaphorin family. In EC, NRP-1 serves as a receptor for some members of the vascular endothelial growth factor (VEGF) family and forms complexes with VEGFR-1 and VEGFR-2 (Neufeld et al). Thus, neuropilins play an important role not only as axon guidance receptors but also in blood vessel development (Eichmann et al., 2005).

Various anti L1-CAM antibodies are known in the art (e.g. mAb 14.10: Huszar et al. 2006; mab chCE7: Meli et al., 1999; mAb UJ127.11: Patel et al., 1991; mAb 5G3: Wolff et al., 1988). It has been suggested in the art to use anti L1-CAM antibodies for the treatment of tumors, especially for the treatment of ovarian and endometrial tumors (cf. US 2004/0259084, US 2004/0115206, and Arlt et al., 2006). Furthermore, it has been suggested to use anti L1-CAM antibodies for sensitizing tumor cells for the treatment with a chemotherapeutic drug or with radiotherapy (Sebens Müerköster et al., 2007), and it has been demonstrated that antibodies directed against L1-CAM synergize with Genistein in inhibiting growth and survival pathways in SKOV3ip human ovarian cancer cells (Novak-Hofer et al., 2008).

There is always a need for further anti-tumor agents.

In a first aspect, the present invention relates to an anti L1-CAM antibody for use in a method for the treatment of a disease in a patient which can be treated by inhibition of angiogenesis, wherein the administration of said anti L1-CAM antibody results in the inhibition of angiogenesis. Furthermore, the present invention also relates to a method for the treatment of a disease in a patient which can be treated by inhibition of angiogenesis, wherein an effective amount of anti L1-CAM antibody is administered to said patient and wherein the administration of said anti L1-CAM antibody results in the inhibition of angiogenesis. Furthermore, the present invention also relates to the use of an anti L1-CAM antibody for the preparation of a pharmaceutical composition for the treatment of a disease in a patient which can be treated by inhibition of angiogenesis, wherein the administration of said anti L1-CAM antibody results in the inhibition of angiogenesis.

In the context of the present invention (see Examples), it has been found that anti L1-CAM antibodies are able to inhibit angiogenesis. Therefore, these antibodies represent a powerful tool for the treatment of diseases which can be treated by inhibition of angiogenesis.

The concept of inhibiting angiogenesis is known in the art (see above). The present invention adds to said concept that also an anti L1-CAM antibody can be used for the inhibition of angiogenesis.

The term “angiogenesis” as used herein may refer to a process of tissue vascularization which involves the formation of new blood vessels. Endothelial cells form new capillaries in vivo when induced to do so, such as during wound repair or in tumor formation or certain other pathological conditions referred to herein as angiogenesis-associated diseases. Angiogenesis may occur via one of the three following mechanisms (Blood and Zetter, 1990):
(1) neovascularization, where endothelial cells migrate out of pre-existing vessels beginning the formation of the new vessels;
(2) vasculogenesis, where the vessels arise from precursor cells de novo; or
(3) vascular expansion, where existing small vessels enlarge in diameter to form larger vessels.

Throughout the invention, the terms “treatment”, “treating”, “treat” and alike generally mean to obtain a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. The term “treatment” as used herein covers any treatment of a disease in a mammal, particularly a human, and includes:
(1) preventing the disease or symptom from occurring in a subject which may be predisposed to the disease or symptom, but has not yet been diagnosed as having it;
(2) inhibiting the disease symptom, i.e., arresting its development; or
(3) relieving the disease symptom, i.e., causing regression of the disease or symptom.

According to the present invention, the term “inhibition” means preferably that the given compound is capable of inhibiting the activity of a respective protein or another substance in the cell at least to a certain amount, e.g. by 50%, 60%, 70%, 80% or 90%. A complete inhibition is also envisaged in the context of the present invention This can be achieved either by a direct interaction of the compound with the given protein or substance (“direct inhibition”) or by an interaction of the compound with other proteins or other substances in or outside the cell which leads to an at least partial inhibition of the activity of the protein or substance (“indirect inhibition”).

Consequently, in the context of inhibition of angiogenesis, the term “inhibition” relates to a process where the administration of the anti L1-CAM antibody results in a reduced angiogenesis when compared to a control where no anti L1-CAM antibody has been administered.

The anti-angiogenic activity of a given anti L1-antibody can be determined by assays known to the person skilled in the art. Especially, an in vitro tube formation assay can be used as described in Example 1 (see below).

In a preferred embodiment of the invention, an anti L1-CAM antibody is used which inhibits angiogenesis by at least about 50%, preferably about 70% or 90%, in an in vitro tube formation assay as described in Example 1.

In a preferred embodiment, the disease which is treated according to the invention by the use of an anti L1-CAM antibody is characterized by a pathological increase and/or induction of angiogenesis.

The term “pathological increase and/or induction” as used herein refers to certain pathological processes in humans where angiogenesis is abnormally prolonged or pathologically induced. Such abnormally prolonged or pathologically induced angiogenesis is found in a variety of angiogenesis-associated diseases including e.g. solid and hematogenous tumors, ocular diseases, chronic inflammatory diseases, especially arthritic diseases, inflammatory skin diseases, neuroinflammatory diseases, Crohn's disease, bartonellosis, transplanted organ rejection, and stomach ulcers. All these diseases as well as others known to the person skilled in the art can be treated in the context of the present invention.

Accordingly, the disease treated by use of an anti L1-CAM antibody according to the present invention is preferably selected from the group consisting of a tumorigenic disease, preferably a solid or a hematogenous tumor, an ocular disease, preferably macular degeneration such as age related macular degeneration, or retinopathy such as proliferative diabetic retinopathy or retinopathy of prematurity, ocular neovascularization, neovascular glaucoma, corneal neovasculature, retinal vein occlusion, or retinal artery occlusion, a chronic inflammatory disease, preferably an arthritic disease, more preferably rheumatoid arthritis, osteoarthritis, psoriatic arthritis, spondyloarthropathies, ankylosing spondylitis, Churg-Strauss syndrome, fibromyalgia, giant cell arthritis, gout, Henoch-Schoenlein Purpura, hypersensitivity vasculitis, polyarthritis nodosa, systemic lupus erythematosus, systemic sclerosis, takayasu arthritis, Wegener's granulomatosis or morbus Reiter, an inflammatory skin disease, preferably dermatitis or psoriasis, a neuroinflammatory disease, preferably multiple sclerosis, Crohn's disease, bartonellosis, transplanted organ rejection, and a stomach ulcer.

In another preferred embodiment of the invention, the disease as defined above is characterized by enhanced expression of L1-CAM on endothelial cells.

In the context of the present invention, the term “endothelial cells” (see also above) means those cells making up the endothelium, the monolayer of simple squamous cells which lines the inner surface of the circulatory system.

The term “enhanced expression” as used herein generally refers to an increased amount of any sort of molecule expressed within a cell or on a cell surface as compared to the amount within a reference cell or on a reference cell surface. In the context of the present invention, the term “enhanced expression of L1-CAM” means the increased amounts of endogenously expressed L1-CAM molecules on the surface of e.g. endothelial cells. Enhanced expression of L1-CAM can be analyzed by means of quantitative immunohistochemical staining using anti L1-CAM antibodies, and can preferably be stimulated by treatment of EC with proinflammatory cytokines such as tumor necrosis factor-α (TNF-α), interferon-γ (INF-γ) or tumor growth factor-β1 (TGF-β1) and/or tumor cell lysates. Alternatively, enhanced expression can e.g. also be determined by RT-PCR.

In a preferred embodiment, the enhanced expression of L1-CAM correlates with increased vascularization of the tissue affected by the disease.

The term “vascularization” as used in the context the present invention means the organic process whereby body tissue becomes vascular and develops capillaries, including the formation of vessels, especially blood vessels.

As shown in the examples, the expression of L1-CAM on endothelial cells enhances tube formation of endothelial cells, which implies a role of said molecule in vascularization.

Although L1-CAM expression in endothelial cells was noted before by immunohistochemical methods, the biological significance of this had not been addressed yet. L1-CAM is cleaved into a soluble form by ectodomain shedding and soluble L1-CAM is able to bind to cells via integrins (Mechtersheimer et al., 2001). So far, it remained unclear whether L1-CAM is endogenously expressed on EC, or whether it is passively acquired in a soluble form from other cells. In the context of the present invention, it has been shown that L1-CAM is endogenously expressed in a full-length form on endothelial cells.

Accordingly, in a preferred embodiment, the anti L1-CAM antibody of the invention binds to L1-CAM expressed on endothelial cells.

In an especially preferred embodiment, the disease treated by use of the anti L1-CAM antibody of the invention is a tumorigenic disease. However, it is equally preferred that the disease is not a tumorigenic disease.

The term “tumorigenic disease” refers to a disease provoked by a tumor (malignant or benign). Throughout the invention, the terms “malignant tumor” and cancer have the same meaning.

In another preferred embodiment of the invention, the anti L1-CAM antibody of the invention binds to L1-CAM on tumor endothelial cells.

Throughout the invention, the term “tumor endothelial cells” denotes endothelial cells within the tumor tissue or in proximity to the tumor tissue. For example, said tumor endothelial cells may be endothelial cells of the blood vessels within the tumor or close by to the tumor.

Consequently, in a further preferred embodiment, said endothelial cells isolated from tumor tissue express higher amounts of L1-CAM as compared to the amount of endogenously expressed L1-CAM molecules on the surface of endothelial cells derived from non-malignant tissue. For pancreatic tumors, it is shown in the examples that tumor endothelial cells express higher amounts of L1-CAM than endothelial cells derived from non-malignant pancreatic tissue.

Preferably, the tumorigenic disease treated by use of an anti L1-CAM antibody of the invention is selected from the group of tumors consisting of astrocytoma, oligodendroglioma, meningioma, neurofibroma, glioblastoma, ependymoma, Schwannoma, neurofibrosarcoma, medulloblastoma, melanoma, preferably malignant melanoma, pancreatic cancer, prostate carcinoma, head and neck cancer, breast cancer, lung cancer, preferably small cell lung cancer, non-small cell lung cancer, colon cancer, preferably adenocarcinoma of the colon, colorectal cancer, gastrointestinal stromal tumor, ovarian cancer, endometrial cancer, renal cancer, neuroblastomas, squamous cell carcinomas, medulloblastomas, hepatoma and mesothelioma, epidermoid carcinoma, clear cell adenocarcinoma and serous adenocarcinoma of the uterine corps, cervix carcinoma, urinary tract adenocarcinoma, Pheochromocytoma, neuroma, neurillemoma, and paranganglioma. According to an especially preferred embodiment, the disease is pancreas carcinoma.

According to another preferred embodiment of the invention, said anti L1-CAM antibody is used in combination with at least one additional therapeutic agent, or in combination with radiotherapy.

Within said combination therapy, it is envisaged that each agent is administered in an effective amount, i.e. that each agent alone is administered in an amount suitable for the treatment of the disease. However, it is also envisaged that each agent is used in subtherapeutic amounts with the consequence that the combination of both agents results in the desired effect.

The term “therapeutic agent” as used herein refers to any drug suitable for the treatment of the respective disease, for example for tumor treatment.

In a preferred embodiment, the anti L1-CAM antibody is used in combination with a chemotherapeutic drug, e.g. selected from the group consisting of actinomycin-D, mitomycin C, cisplatin, doxorubicin, etoposide, verapamil, podophyllotoxin, 5-FU, taxans such as paclitaxel, and carboplatin, or selected from the group consisting of
(i) an alkylating agent including busulfan, carmustine, chlorambucil, cyclophosphamide (i.e., cytoxan), dacarbazine, ifosfamide, lomustine, mecholarethamine, melphalan, platinum containing compounds like cisplatin and carboplatin, procarbazine, streptozocin, and thiotepa, preferably platinum containing compounds like cisplatin and carboplatin,
(ii) an antineoplastic agent including antimitotic agents like paclitaxel or a derivative thereof, bleomycin, dactinomycin, daunorubicin, doxorubicin, idarubicin, mitomycin (e.g., mitomycin C), mitoxantrone, pentostatin, and plicamycin, preferably antimitotic agents like paclitaxel or a derivative thereof,
(iii) an RNA/DNA antimetabolite including fluorodeoxyuridine, capecitabine, cladribine, cytarabine, floxuridine, fludarabine, fluorouracil, gemcitabine, hydroxyurea, mercaptopurine, methotrexate, and thioguanine, preferably 5-fluorouracil (5FU) or capecitabine,
(iv) a natural source derivative including docetaxel, etoposide, irinotecan, paclitaxel, teniposide, topotecan, vinblastine, vincristine, vinorelbine, taxol, prednisone, and tamoxifen, and
(v) an additional chemotherapeutic agent including asparaginase, mitotane, leucovorin, oxaliplatin, DNA topoisomerase inhibiting agents like camptothecin, and anthracyclines like doxorubicin.

Preferably, the chemotherapeutic agent is or comprises oxaliplatin and/or irinotecan.

The term “radiotherapy” as used herein further refers to each radiation therapy which is commonly used to treat tumors cells. In a preferred embodiment, this therapy include γ-rays, X-rays, microwaves, UV radiation as well as the direct delivery of radio-isotopes to or next to tumor cells (brachytherapy).

According to the present invention, the term “in combination with” includes any combined administration of an anti L1-CAM antibody with either the therapeutic agent or the treatment of radiotherapy. This may include the simultaneous application of the therapeutic agent or radiotherapy or, preferably, a separate administration. In case that a separate administration is envisaged, one would preferably ensure that a significant period of time would not expire between the timepoint of delivery, such that the anti L1-CAM antibody of the invention and the therapeutic agent or the radiotherapy would still be able to exert an advantageously combined effect on the cell. In such instances, it is preferred that one would contact the cell with both agents within a time period of about one week, preferably within about 4 days, more preferably within about 12-36 hours of each other. Furthermore, this aspect of the invention also encompasses treatment regimens where the anti L1-CAM antibody according to the invention is administered in combination with an therapeutic agent or radiotherapy in various treatment cycles wherein each cycle may be separated by a period of time without treatment which may last e.g. for two weeks and wherein each cycle may involve the repeated administration of the anti L1-CAM antibody and/or the therapeutic agent or radiotherapy. For example such treatment cycle may encompass the treatment with a therapeutic agent or with radiotherapy, followed by e.g. the twice application of the anti L1-CAM antibody within 2 days.

Consequently, this includes that the antibody may be administered before, in parallel or after the therapeutic agent or the radiotherapy.

In another preferred embodiment of the invention, the additional therapeutic agent is an antiangiogenic agent.

The term “antiangiogenic agent” as used herein in general refers to a substance, a composition, a drug, or a chemical reagent, or alike which functions as an angiogenesis inhibitor, i.e. which inhibits the growth of new blood vessels.

According to the present invention, an antiangiogenic agent is preferably an agent which is selected from the group consisting of agents that target the vascular epidermal growth factor (VEGF) pathway, the platelet derived growth factor 1 (PDGF1), endothelial growth factor (EGF), or fibroblast growth factor (FGF) pathway, an integrin, a matrix metalloproteinase (MMP) and/or protein kinase C beta (PKCβ) inhibitor, or a combination thereof. Vascular endothelial growth factor (VEGF)-mediated angiogenesis is thought to play a critical role in tumor growth and metastasis. Consequently, anti-VEGF therapies may be anti-cancer treatments, either as alternatives or adjuncts to conventional chemo or radiation therapy. Several approaches to targeting VEGF have been investigated. The most common strategies have been receptor-targeted molecules and VEGF-targeting molecules. Therefore, preferably, said VEGF pathway targeting agent is:
i) an antibody or a fragment thereof against a member of the VEGF family (VEGF, placental growth factor (P1GF), VEGF-B, VEGF-C, VEGF-D) or their receptors (VEGFR-1 (Flt-1), -2 (Flk-1/Kdr), -3 (Flt-4)), and/or
ii) a small molecule tyrosine kinase inhibitor of VEGF receptors, and/or
iii) a soluble VEGF receptor, and/or iv) a ribozyme which specifically targets VEGF mRNA.

In another preferred embodiment of the invention, the antiangiogenic agent targeting an MMP or an integrin is a chimeric, humanized or fully human monoclonal antibody.

The humanized monoclonal antibody Bevacizumab (Avastin™, Genentech) is approved as an anti-angiogenic agent for treatment of cancer. Bevacizumab is preferably administered to human patients intravenously, and is usually administered in an intravenous infusion of 5 mg/kg every 14 days. The therapy is usually not initiated for at least 28 days following major surgery. It is recommended that the surgical incision is fully healed prior to initiation of bevacizumab therapy (Avastin IV in PDR 60. edition, 2006, Thomson, pp. 1229-1232). The monoclonal antibody fragment Ranibizumab (Lucentis™, Genentech) and the aptamer drug Pegaptanib (Macugen™, Pfizer) are approved for the treatment of AMD.

According to a another preferred embodiment of the invention, the antiangiogenic agents which targets an MMP is selected from the group consisting of marimastat, metastat (COL-3), BAY-129566, CGS-27023, prinomastat (AG-3340), and BMS-27529. These drugs are all in different stages of clinical development, ranging from phase I to III.

In another embodiment, the antiangiogenic agent target targets a tyrosine kinase growth factor receptor, e.g. sunitinib or sorafenib.

In an alternative preferred embodiment, the antiangiogenic agents targeting an integrin is selected from the group consisting of SB-267268, JSM6427, and EMD270179.

In yet another alternative preferred embodiment, antiangiogenic agent is selected from the group consisting of a cationic liposome, a Vascular Targeting Agent (VTA), Neovastat (AE-941), U-995, Squalamine, Thalidomide or one of its immunomodulatory analogs, or a combination thereof.

In another aspect, the present invention relates to an anti L1-CAM antibody for use in a method for preventing the formation of a tumor metastasis in a patient. Furthermore, the present invention relates to a method for preventing the formation of a tumor metastasis in a human patient, wherein an effective amount of said anti L1-CAM antibody is administered to said patient. Furthermore, the invention also relates to the use of an anti L1-CAM antibody for the preparation of a pharmaceutical composition for preventing the formation of a tumor metastasis in a patient.

In the context of the present invention, it has been found that anti L1-CAM antibodies are able to prevent transmigration of. tumor cells through endothelial cells, which is an important step in the formation of metastasis.

The term “prevention”, “preventing”, or “prevent” or alike as used herein refers to an at least partial inhibition of the formation of metastasis.

The term “tumor metastasis” as used herein refers to the formation of a so called secondary or metastatic tumor by the spread of malignant tumor cells. Formation of tumor metastasis may be characterized by the circulation of tumor cells through the bloodstream and migration through lymphatic and blood vessels, before they settle down to grow within normal tissues elsewhere in the body. Most tumors and other neoplasms can metastasize, although in varying degrees.

In a preferred embodiment, this prevention is mediated by inhibition of tumor cell adhesion to endothelial cells and/or tumor cell transmigration over endothelial cells.

One step in the process of metastasis may be the attachment of tumor cells to endothelial cells (EC). The term “cell adhesion” as used herein refers to the binding of a cell to another cell or to a surface or matrix. Cellular adhesion can be regulated by specific cell adhesion molecules that interact with molecules on the opposing cell or surface. Human cells have many different types of adhesion molecules, and the major classes are named integrins, Ig superfamily members, cadherins, and selectins. Each of these adhesion molecules has a different function and recognizes different ligands and/or receptors.

The term “cell transmigration” as used herein refers to process of enabling a cell to migrate out of the bloodstream into a tissue or out of the tissue into the bloodstream. Transmigration of a cell may be mediated by cell-cell adhesion and thus by the action of e.g. selectins and/or integrins and their respective receptors. In the context of the present invention, cell transmigration preferably means the migration of tumor cells into a tissue as a prerequisite for tumor metastasis formation is said tissue.

In a preferred embodiment, the inhibition of tumor cell adhesion to endothelial cells and/or the inhibition of tumor cell transmigration over endothelial cells correlate with enhanced expression of L1-CAM on endothelial cells.

In another specific preferred embodiment, said endothelial cells are tumor endothelial cells. The term “tumor endothelial cells” has already been defined above.

In the context of the present invention (see Examples), the inventors have found that (i) L1-CAM is up-regulated on tumor endothelial cells, that (ii) the interaction between tumor endothelial cells can be inhibited by anti L1-CAM antibodies and that (iii), consequently, transmigration of tumor cells through said tumor endothelial cells can be inhibited. Therefore, anti L1-CAM antibodies represent a powerful tool for the prevention of metastasis.

Therefore, in this preferred embodiment of the invention, the formation of metastasis is prevented by inhibiting transmigration of tumor cells though endothelial cells of the tumor tissue, e.g. to the blood vessel.

However, it is also envisaged within the present invention that transmigration of tumor cells from blood vessels in another tissue where the metastasis is subsequently formed can be inhibited by an anti L1-CAM antibody of the invention.

In another preferred embodiment, the tumor metastasis originates from a tumor which is selected from the group of tumors consisting of astrocytoma, oligodendroglioma, meningioma, neurofibroma, glioblastoma, ependymoma, Schwannoma, neurofibrosarcoma, medulloblastoma, melanoma, preferably malignant melanoma, pancreatic cancer, prostate carcinoma, head and neck cancer, breast cancer, lung cancer, preferably small cell lung cancer, non-small cell lung cancer, colon cancer, preferably adenocarcinoma of the colon, colorectal cancer, gastrointestinal stromal tumor, ovarian cancer, endometrial cancer, renal cancer, neuroblastomas, squamous cell carcinomas, medulloblastomas, hepatoma and mesothelioma, epidermoid carcinoma, clear cell adenocarcinoma and serous adenocarcinoma of the uterine corps, cervix carcinoma, urinary tract adenocarcinoma, Pheochromocytoma, neuroma, neurillemoma, and paranganglioma. In a more preferred embodiment, the tumor metastasis preferably originates from a pancreas carcinoma.

In all aspects of the present invention, an anti L1-CAM antibody is used for disease treatment.

The preparation of anti L1-CAM antibodies has been described in the art (see, e.g., Huszar et al., 2006; Mujoo et al., 1986; Gast et al., 2008).

Furthermore, anti L1-CAM antibodies are also commercially available. Examples of commercially available antibodies are, e.g., UJ127.11 (Santa Cruz Biotech, Cat. No. sc-53386) or 5G3 (BD Pharmingen, Cat. No. 5542). Other suppliers are Signet or Abcam

In the context of the present invention, the term “antibody” refers to all known sorts of recombinantly or synthetically generated/synthesized antibodies including full immunoglobulin molecules, preferably IgMs, IgDs, IgEs, IgAs or IgGs, more preferably IgG1, IgG2a, IgG2b, IgG3 or IgG4, as well as antibody fragments like Fab-fragments or VL-, VH- or CDR-regions, antigen-binding parts thereof and/or binding molecules, which have been, where appropriate, modified, such as chimeric antibodies, humanized antibodies, multifunctional antibodies, bispecific or oligospecific antibodies, single-stranded antibodies and F(ab) or F(ab)₂ fragments (see, e.g, U.S. Pat. No. 6,248,516, U.S. Pat. No. 4,816,567). The procedure for preparing an antibody or antibody fragment is performed in accordance with methods that are well know to a person skilled in the art, such as immunization of a mammal, e.g. a rabbit, with a protein or peptide of interest and, where appropriate, e.g. in the presence of Freund's adjuvant and/or aluminium hydroxide gels (see, for example, Diamond, B. A. et al. (1981) The New England Journal of Medicine: 1344-1349).

In a preferred embodiment of the invention, the antibody is selected from the group consisting of single chain antibodies (e.g. scFv or (scFv)₂, antibody fragments (e.g. Fab), tandabs, diabodies, flexibodies, bispecific antibodies, or chimeric antibodies.

Antibodies can be raised against different domains of a protein of interest. In the context of the present invention, antibodies targeting L1-CAM can, for example, been raised against one or more of the six immunoglobulin-like domains (1 to 6), against one or more of the five fibronectin (FN)-type III repeats, against the highly conserved cytoplasmic tail, or against a combination thereof.

In a preferred embodiment of the present invention, the antibody is a polyclonal or a monoclonal antibody.

The term “polyclonal” as used herein refers to antibodies which are formed in the animal as a result of an immunological reaction, and which can subsequently be isolated from the blood using well known methods and, for example, be purified by means of column chromatography. In detail, polyclonal antibodies can be prepared by immunizing a suitable subject with a polypeptide as an immunogen. Preferred polyclonal antibody compositions are ones that have been selected for antibodies directed against a polypeptide or polypeptides of the invention, e.g. L1-CAM or a fragment thereof. Particularly preferred polyclonal antibody preparations are ones that contain only antibodies directed against a given polypeptide or polypeptides. Particularly preferred immunogen compositions are those that contain no other human proteins such as, for example, immunogen compositions made using a non-human host cell for recombinant expression of a polypeptide of the invention.

The term “monoclonal” as used herein refers in general to monoclonal antibodies which can, for example, be prepared in accordance with the known method of Winter and Milstein (Winter, G. and Milstein, C. (1991) Nature, 349, 293-299). Alternatively, monoclonal antibodies can be produced by antibody-secreting hybridomas, or can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612).

Antibodies can be raised against different domains of a protein of interest. In the context of the present invention, antibodies against L1-CAM can either specifically target one of the six immunoglobulin-like domains (1 to 6), or a combination thereof, or they can specifically recognize one of the five fibronectin (FN)-type III repeats. According to a preferred embodiment of the invention, the epitope of the antibody used in the context of the invention is within the immunoglobulin-like domains of L1-CAM. According to an especially preferred embodiment of the present invention, the epitope of the antibody used in the context of the invention is within the first immunoglobulin-like domain of L1-CAM.

The effect of an antibody is mediated by its capacity to bind a specific epitope. In the context of the present invention, the epitope resides on L1-CAM. Methods for determining the epitope of a given antibody are known in the art and include the preparation of synthetic linear peptides of a given region of interest and the subsequent testing whether the antibody binds to said peptides. Alternatively, different recombinant proteins covering the region of interest can be produced and tested for the binding of the antibody (Oleszewski et al., 2000).

Preferably, the anti L1-CAM antibody used in the context of the present invention is capable of binding to the same L1 epitope recognized by the monoclonal antibody 9.3, produced by the hybridoma cell deposited under DSMZ ACC2841.

In another preferred embodiment, the anti L1-CAM antibody has the same capacity to inhibit tumor growth as the monoclonal antibody 9.3, produced by the hybridoma cell deposited under DSMZ ACC2841. According to the invention, “the same capacity” means that the monoclonal antibody has a tumor growth inhibiting capacity which does not differ more than 5% from the tumor growth inhibiting capacity of the monoclonal antibody 9.3.

Preferably, the monoclonal antibody is produced by the hybridoma cell deposited under DSMZ ACC2841. This hybridoma cell has been deposited with the Deutsche Sammlung für Mikroorganismen and Zellen on Apr. 25, 2007 under the Budapest Treaty.

In another preferred embodiment, the an anti L1 monoclonal antibody used in the context of the present invention is characterized in that at least one of its complementarity determining regions (CDRs)
a) has one of the following sequences RASQDISNYLN, YTSRLHS, QQGNTLPWT, RYWML, EINPRNDRTNYNEKFKT, or GGGYAMDY or
b) has a sequence which, in comparison to the sequences mentioned under a) has at least one conservative amino acid exchange.

The above mentioned sequences show the CDRs of the monoclonal antibody 9.3 determined according to the method of Kabat. Such a monoclonal antibody of the invention can, e.g., be produced by CDR grafting or by recombinant production of the antibody. Such methods are known in the art (see, e.g., Queen, U.S. Pat. No. 5,585,089, Winter, U.S. Pat. No. 5,225,539, and Cabilly U.S. Pat. No. 4,816,567).

In another aspect, the antibody used according to the invention is humanized.

Humanized antibodies are antibody molecules from non-human species having one or more complementarily determining regions (CDRs) from the non-human species and a framework region (FR) from a human immunoglobulin molecule. (see, e.g., Queen, U.S. Pat. No. 5,585,089 and Winter, U.S. Pat. No. 5,225,539.) Such chimeric and humanized monoclonal antibodies can be produced by recombinant DNA techniques known in the art.

In general, in order to obtain a humanized antibody, nucleic acid sequences encoding human variable heavy chains and variable light chains may be altered by replacing one or more CDR sequences of the human (acceptor) sequence by sequence encoding the respective CDR in the mouse antibody sequence (donor sequence). The human acceptor sequence may comprise FR derived from different genes.

In a preferred embodiment, the humanized antibody has at least one non-human CDR and human framework region (FR) residues.

Sequences encoding full length antibodies can be subsequently obtained by joining the rendered variable heavy and variable light chain sequences to human constant heavy chain and constant light chain regions. Preferred human constant light chain sequences include kappa and lambda constant light chain sequences. Preferred human constant heavy chain sequences include IgG1, IgG2 and sequences encoding IgG1 mutants which have rendered immune-stimulating properties.

Suitable human donor sequences can be determined by sequence comparison of the peptide sequences encoded by the mouse donor sequences to a group of human sequences, preferably to sequences encoded by human germ line immunoglobulin genes or mature antibody genes. A human sequence with a high sequence homology, preferably with the highest homology determined may serve as the acceptor sequence for the humanization process.

In addition to the exchange of human CDRs for mouse CDRs, further manipulations in the human donor sequence may be carried out to obtain a sequence encoding a humanized antibody with optimized properties (such as affinity of the antigen).

The altered human acceptor antibody variable domain sequences may be rendered to encode one or more amino acids (according to the Kabat numbering system) of position 4, 35, 38, 43, 44, 46, 58, 62, 64, 65, 66, 67, 68, 69, 73, 85, 98 of the light variable region and 2, 4, 36, 39, 43, 45, 69, 70, 74, 75, 76, 78, 92 of the heavy variable region corresponding to the mouse donor sequence (Carter and Presta, U.S. Pat. No. 6,407,213). Also the sequences of the CDRs may be altered, preferably by exchanges leading to a conservative amino acid exchange.

In general, manipulations may result in alterations in the FR as well as the CDR regions and include exchanges, deletions and insertion of residues. The alterations may be induced by random or directed mutagenesis. A antibody phage display system, as described before, may be employed for the selection of mutants with desired and/or improved properties

Furthermore, the antibody used in the context of the present invention may be a human antibody capable of recognizing the same epitope as the antibody 9.3. Methods for generating human antibodies are known in the art. These methods employ for example mice in which the endogenous immunoglobuline genes have been partially or completely inactivated and human immunoglobuline loci were introduced. Upon immunization with an immunogenic epitope, these mice are capable of producing human antibodies (U.S. Pat. Nos. 5,545,807; 5,545,806; 5,569,825; 5,589,369; 5,591,669; 5,625,126; 5,633,425; 5,661,016)

It is another aspect of the present invention that other L1-CAM-interfering molecules can be used interchangeably for anti L1-CAM antibodies described in the present invention.

In the context of the present invention, the term “L1-CAM-interfering molecules” as used herein refers to molecules which interfere with the biological function of L1-CAM.

L1-CAM-interfering molecules can be L1-CAM-binding molecules which are capable of binding to L1-CAM either extracellularly (e.g. an antibody as discussed above, an anticalin or an aptamer), or intracellularly (e.g. a low molecular weight molecule).

Furthermore, the term “L1-CAM-interfering molecule” may relate to a nucleic acid which is complementary to L1-CAM coding sequences in the endothelial cell, e.g. DNA or mRNA encoding L1-CAM or parts thereof, and which modulates, and preferably inhibits L1-CAM expression in the endothelial cell when entering an endothelial cell. Such molecules are discussed below with reference to siRNA, antisense molecules and ribozymes

According to the invention, such inhibition may be complete or partial, e.g. the expression may be reduced by at least 50% or by at least 80%.

Furthermore, the term “L1-CAM-interfering molecule” also relates to molecules which act in a downstream activity cascade triggered by L1-CAM. This includes e.g. molecules that bind to protein kinases activated upon binding of a ligand to L1-CAM.

In a preferred embodiment, a L1-CAM-interfering molecule according to this aspect of the invention is a molecule as defined above which is capable of inhibiting tube formation in an in vitro tube formation assay such as described in Example 1.

An aptamer is a RNA, DNA or peptide molecule that binds to a specific target molecule.

The production and use of siRNAs as tools for RNA interference in the process to down regulate or to switch off gene expression, here L1-CAM gene expression, is e.g. described in Elbashir et al. (2001). Preferably, siRNAs exhibit a length of less than 30 nucleotides, wherein the identity stretch of the sense strand of the siRNA is preferably at least 19 nucleotides.

An “antisense” nucleic acid as used herein refers to a nucleic acid capable of hybridizing to a sequence-specific portion of a component protein RNA (preferably mRNA) by virtue of some sequence complementarity. The antisense nucleic acid may be complementary to a coding and/or noncoding region of a component protein mRNA.

The antisense nucleic acids are of at least six nucleotides and are preferably oligonucleotides, ranging from 6 to about 200 nucleotides. In specific aspects, the oligonucleotide is at least 10 nucleotides, at least 15 nucleotides, at least 100 nucleotides, or at least 200 nucleotides.

Ribozymes are also suitable tools to inhibit the translation of nucleic acids, because they are able to specifically bind and cut the mRNAs. They are e.g. described in Vaish et al. (1998) Nucleic Acids Res., 26, 5237-42.

LMW molecules (low molecular weight molecules) are molecules which are not proteins, peptides, antibodies or nucleic acids, and which exhibit a molecular weight of less than 5000 Da, preferably less than 2000 Da, more preferably less than 1000 Da, most preferably less than 500 Da. Such LMWs may be identified in High-Through-Put procedures starting from libraries.

Throughout the present invention, the anti L1-CAM antibody may be formulated to a pharmaceutical composition.

Preferably, the antibody may be formulated, in accordance with routine procedures, as a pharmaceutical composition adapted for intravenous administration to human beings, although also other administration routes are envisaged (see below). Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline for injection can be provided so that the ingredients may be mixed prior to administration.

The antibody may further be formulated as neutral or salt forms. Pharmaceutically acceptable salts include those formed with free carboxyl groups such as those derived from hydrochloric, phosphoric, acetic, oxalic, tartaric acids, etc., those formed with free amine groups such as those derived from isopropylamine, triethylamine, 2-ethylamino ethanol, histidine, procaine, etc., and those derived from sodium, potassium, ammonium, calcium, and ferric hydroxides, etc.

The amount of the antibody of the invention, which will be effective in the treatment of a particular disorder or condition, will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems. In general, suppositories may contain active ingredient in the range of 0.5% to 10% by weight; oral formulations preferably contain 10% to 95% active ingredient.

Various delivery systems are known and can be used to administer a therapeutic of the invention, e.g., encapsulation in liposomes, microparticles, and microcapsules: use of recombinant cells capable of expressing the therapeutic, use of receptor-mediated endocytosis (see, e.g., Wu and Wu, 1987, J. Biol. Chem. 262:4429-4432); construction of a therapeutic nucleic acid as part of a retroviral or other vector, etc. Methods of introduction include but are not limited to intradermal, intramuscular, intraperitoneal, intravenous, subcutaneous, intranasal, epidural, and oral routes. The compounds may be administered by any convenient route, for example by infusion, by bolus injection, by absorption through epithelial or mucocutaneous linings (e.g., oral, rectal and intestinal mucosa, etc.), and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

Throughout the invention, the patient is preferably a mammal, and more preferably a human patient.

The invention is further illustrated by the following figures and examples which are not intended to limit the scope of the invention.

### SHORT LEGENDS TO THE FIGURES

FIG. 1. Enhanced L1-CAM Expression in Non-Malignant Pancreatic Tissue and Pancreatic Carcinomas

(A) Cryosections (5 μm) of pancreatic tumour tissue (Tu) or corresponding non-malignant pancreatic tissue (Con) were stained with monoclonal antibodies to L1-CAM (red) and CD31 (green) or with respective isotype antibodies (bottom left). Nuclei were counterstained with 4′,6-diamidino-2-phenylindol (blue). Original magnification 400× (top left and bottom panel) and 630× (top right). Arrows indicate L1-CAM-CD31 co-localization. (B) Quantification of total L1-CAM expression and L1-CAM expressing endothelium in primary pancreatic carcinoma tissue (Tu, grey bars) and nonmalignant pancreas tissue (Con, black bars). Total percentage of CD31- and L1-CAM-expressing endothelium per mm² tissue was determined by immunohistology on frozen tissue sections. Means (±SD) of 8-10 tissue samples from 24 independent donors with evaluation of 3-5 sections per sample are shown. ** P<0.05 (two-sided student t test).

FIG. 2. Regulation of L1-CAM Expression by Cultured ECs by Tumour Cell Lysate and Proinflammatory Cytokines

(A) Expression of L1-CAM (red) on cultured non-malignant endothelial cells (Con HUVEC, left, Con HPMEC, middle) and tumour-derived endothelial cells (TuPAMEC, right) co-cultured with 50 μg/ml tumour cell lysates (Panc1 lysate, middle panel) or proinflammatory cytokine TNF-α (400 U/ml, bottom panel). Nuclei were counterstained with 4′,6-diamidino-2-phenylindol (blue). Original magnification 400×. (B) Flow cytometric analysis of L1-CAM expression on cultured non-malignant ECs (Con HUVEC, left, Con HPMEC, middle) and TuPAMEC (right) untreated (dark grey histograms) or stimulated for 24 h with proinflammatory cytokines TNF-α (400 U/ml, dotted line), IFN-γ (1000 U/ml, dashed line) and TGF-β1 (10 ng/ml, dashed-dotted line). Light grey histograms represent negative control staining with respective secondary antibodies. (C) Immunocytologic quantification of induced L1-CAM expression of non-malignant endothelial cells (Con HUVEC, left, Con HPMEC, middle) after incubation with 50 μg/ml tumour cell lysate (Panc1 lysate) or 50 μg/ml non tumour cell lysates as control (PBMC lysate, HEK293L1 lysate) for 72 h. Means (±SD) of three independent experiments performed in triplicates with 3-5 sections per sample are shown. ** P<0.05 (two-sided student t test). (D) Immunocytologic quantification of induced L1-CAM expression on non-malignant endothelial cells (Con HUVEC, white bars, Con HPMEC, grey bars) and TuPAMEC (black bars) after incubation with proinflammatory cytokines TNF-α, IFN-γ and TGF-β1 for 24 h. Means (±SD) of three independent experiments performed in triplicates with 3-5 sections per sample are shown. ** P<0.05 (two-sided student t test).

FIG. 3. Role of L1-CAM and NRP-1 in the Transmigration Through EC Monolayers

(A) Basic transmigration of non-malignant endothelial cells (Con HUVEC, white bars) and tumour-derived endothelial cells (Tu PAMEC, black bars) without chemokine stimulus. Endothelial cells were added in three different concentrations to transmigration chambers (8 μm pores). After 24 h transmigrated cells in the lower chamber were counted. Mean cell numbers (±SD) of three independent experiments each performed in triplicates are shown. * P<0.1, ** P<0.05 (two-sided student test). (B) Inhibition of SDF1-β stimulated transmigration of non-malignant endothelial cells (Con HUVEC, white bars, Con HPMEC, grey bars, 10⁵/100 μl cells per well) and tumour-derived endothelial cells (TuPAMEC, black bars, 10⁵/100 μl cells per well) after blocking of L1-CAM and NRP-1 by respective monoclonal antibodies. Mean relative inhibition (±SD) of transmigration compared to blocking by respective isotype control. ** P<0.05 (two-sided student t test).

FIG. 4. Essential Role of L1-CAM and NRP-1 for Endothelial Cell Tube Formation

(A) Tube formation on matrigel of non-malignant EC (Con HUVEC, upper panel) and tumour-derived EC (TuPAMEC, bottom panel). Inhibition of tube formation of non-malignant EC (Con HUVEC, 2×10⁴/1000) and tumour-derived endothelial cells (TuPAMEC, 2×10⁴/100 μl) after blocking of L1-CAM (anti L1-CAM mAb, middle right) and NRP-1 (anti NRP-1 mAb, right) by respective monoclonal antibodies are shown. Respective isotype antibody was used for specificity control (isotype control, middle left). Original magnification 50×. Numbers in bottom line indicate quantification of basic tube formation on matrigel after 24 h of tumour-derived EC (Tu PAMEC) and non-malignant EC (Con HUVEC). Mean (±SD) of three independent experiments each performed in triplicates are shown. ** P<0.05 (two-sided student t test). (B) Relative inhibition of tube formation of tumour-derived EC (Tu PAMEC, black bars) and non-malignant EC (Con HUVEC, white bars, Con HPMEC, grey bars) after blocking of L1-CAM (anti L1-CAM mAb) and NRP-1 (anti NRP-1 mAb) by monoclonal antibodies compared to respective isotype antibody (isotype control). Mean (±SD) of three independent experiments each performed in triplicates are shown. ** P<0.05 (two-sided student t test). (C) Relative inhibition of tube formation of non-malignant EC (Con HUVEC, white bars) after incubation with 50 μg/ml tumour cell lysate (Panc1 lysate, generated by freezing and thawing cycles, black bars) or 50 μg/ml non tumour cell lysates as control (PBMC lysate, light grey bars, HEK293L1 lysate, dark greys bars) for 72 h and subsequent blocking of L1-CAM (anti L1-CAM mAb) and NRP-1 (anti NRP-1 mAb) by respective monoclonal antibodies. Respective isotype antibody was used for specificity control (isotype control). Mean (±SD) of three independent experiments each performed in triplicates are shown. ** P<0.05 (two-sided student t test).

FIG. 5. Functional Properties of L1-CAM and NRP-1 on ECs for Panc1 Tumour Cell Adhesion

(A) Cell adhesion of carboxyfluorescein diacetate succinimidyl ester (CFSE) stained Panc1 tumour cells to tumour-derived endothelial cell monolayer (Tu PAMEC, right) and non-malignant endothelial cell monolayer (Con HUVEC, left, Con HPMEC, middle). Immunocytologic quantification of relative adhesion of Panc1 tumour cells to tumour-derived endothelial cell monolayer (Tu PAMEC, black bars) and non-malignant endothelial cell monolayer (Con HUVEC, white bars, Con HPMEC, grey bars) is shown. Means (±SD) of three independent experiments performed in triplicates with 5-8 sections per sample are shown. ** P<0.05, *** P<0.001 (two-sided student t test). (B) Inhibition of Panc1 tumour cell adhesion to tumour-derived EC monolayer (Tu PAMEC, bottom panel) and non-malignant EC monolayer (Con HUVEC, upper panel, Con HPMEC, middle panel) after blocking of L1-CAM (anti L1-CAM mAb) and NRP-1 (anti NRP-1 mAb) by respective monoclonal antibodies. Respective isotype antibody was used as specificity control (isotype control). (C) Relative inhibition of Panc1 tumour cell adhesion to tumour-derived EC monolayer (black bars) and non-malignant EC monolayer (Con HUVEC, white bars, Con HPMEC, grey bars) after selective EC blocking (left panel) or tumour cell blocking (right panel) of L1-CAM (anti L1-CAM mAb) and NRP-1 (anti NRP-1 mAb) by respective monoclonal antibodies. Isotype antibody (isotype control) was used as specificity control. Means (±SD) of three independent experiments performed in triplicates are shown. ** P<0.05, *** P<0.001 (two-sided student t test).

FIG. 6. Analysis of Panc1 Tumour Cell Transmigration Through Endothelial Cell Monolayers

Tumour-derived endothelial cells (TuPAMEC, black bars, 2×10⁵/100 μl) and non-malignant endothelial cell monolayer (Con HUVEC, white bars, 2×10⁵/100 μl, Con HPMEC, grey bars, 2×10⁵/100 μl) were cultured for 48 h on gelatin-coated transmigration membranes (5 μm pores) until confluency and for the last 24 h activated with TNF-α. Subsequently, Panc1 tumour cells were added (1×10⁵/100 μl). Transmigrated Panc1 tumour cells were counted after 24 h. Three independent experiments each performed in triplicates are shown. (A) Relative inhibition of SDF-1β stimulated transmigrated Panc1 tumour cells through tumour-derived endothelial cells (TuPAMEC, black bars) and non-malignant endothelial cell monolayer (Con HUVEC, white bars, Con HPMEC, grey bars) after selective endothelial cell blocking of L1-CAM (anti L1-CAM mAb) and NRP-1 (anti NRP-1 mAb) by respective monoclonal antibodies. Isotype antibody (isotype control) was used as specificity control. Mean (±SD) is shown. ** P<0.05 (two-sided student t test). (B) Relative inhibition of SDF-1β stimulated transmigrated Panc1 tumour cells through tumour-derived endothelial cells (TuPAMEC, black bars) and non-malignant endothelial cell monolayer (Con HUVEC, white bars, Con HPMEC, grey bars) after selective tumour cell blocking of L1-CAM (anti L1-CAM mAb) and NRP-1 (anti NRP-1 mAb) by respective monoclonal antibodies. Isotype antibody (isotype control) was used as specificity control. Mean (±SD) of three independent experiments each performed in triplicates is shown.

FIG. 7. Transendothelial Migration of Isolated Suppressive Regulatory T Cells (Treg) Through Endothelial Monolayers

Treg cells were isolated from peripheral blood samples by depletion of non-CD4⁺ T cells and subsequently positive isolation using CD25 magnetic MicroBeads. Isolated Tregs were characterized by flow cytometry as CD3⁺, CD4⁺, CD25⁺ and FoxP3⁺ cells. (A) Flow cytometric analysis of L1-CAM expression of isolated Treg cells (dotted line). Tregs were gated as CD3⁺, CD4⁺, CD25⁺ and FoxP3⁺ cells. Light grey histogram represents negative control staining with respective isotype antibody. (B) Quantitative RT-PCR analysis of L1-CAM expression of isolated Treg and Tcon cells. Three independent experiments each performed in triplicates were performed. Means (±SD) from one of three representative experiments are shown. (C) Tumour-derived endothelial cells (TuPAMEC, black bars, 2×10⁵/100 μl) and non-malignant endothelial cell monolayer (Con HUVEC, white bars, 2×10⁵/100 μl, Con HPMEC, grey bars, 2×10⁵/100 μl) were cultured for 48 h on gelatin-coated transmigration membranes (5 μm pores) until confluency and for the last 24 h activated with TNF-α. Subsequently, Tcon (white bars, 1×10⁵/100 μl) or Treg cells (black bars, 1×10⁵/100 μl) were added. SDF-1β stimulated transmigrated T cells were counted after 24 h. Mean (±SD) of three independent experiments each performed in triplicates are shown. ** P<0.05 (two-sided student t test). (D) Relative inhibition of SDF-1β stimulated transmigration of Treg cells through tumour-derived endothelial cell monolayer (TuPAMEC) and non-malignant endothelial cell monolayer (Con HUVEC) after selective blocking of L1-CAM (anti L1-CAM mAb, black bars) by respective monoclonal antibody. Respective isotype antibody was used as specificity control (grey bars). Mean (±SD) of three independent experiments each performed in triplicates are shown. ** P<0.05 (two-sided student t test). (E) Transmigration of Panc1 tumour cells (1×10⁵/100 μl) through non-malignant endothelial cell monolayer (Con HUVEC) is shown. For stimulation, no cells (white bar), Tcon (1×10⁴/500 μl, 5×10⁴/500 μl and 1×10⁵/500 μl, grey bars) or Treg cells (1×10⁴/500 μl, 5×10⁴/500 μl and 1×10⁵/500 μl, black bars) were added in the lower chamber. Transmigrated tumour cells were counted after 24 h. Mean (±SD) of three independent experiments each performed in triplicates are shown. ** P<0.05 (two-sided student t test). (F) Relative migration of Panc1 tumour cells through non-malignant endothelial cell monolayer (Con HUVEC, black bars) counted after 24 h. Transmigration was not stimulated (white bar) or TGF-β1 stimulated (1 ng/ml, 10 ng/ml and 100 ng/ml). Mean (±SD) of three independent experiments each performed in triplicates are shown. ** P<0.05 (two-sided student t test).

FIG. 8. Analysis of Conditions for Achieving Confluent Endothelial Monolayers

Hematoxylin and eosin staining of cultured non-malignant EC (Con HUVEC) on a gelatine coated membranes. Different conditions (1×105/24 h, left panel and 2×105/48 h, right panel) were tested to obtain a confluent EC monolayer. Original magnification 400×.

FIG. 9. Regulation of NRP-1 Expression of Cultured EC by Proinflammatory Cytokines

Flow cytometric analysis of NRP-1 expression on cultured non-malignant ECs (Con HUVEC, left; Con HPMEC, middle) and TuPAMEC (right) untreated (dark grey histograms) or stimulated for 24 h with proinflammatory cytokines TNF-α (400 U/ml, dotted line), IFN-γ (1000 U/ml, dashed line) and TGF-β1 (10 ng/ml, dashed-dotted line). Light grey histograms represent negative control staining with respective secondary antibodies.

FIG. 10. Measurement of T cell Proliferation Inhibition by Activated Treg Cells

Measurement of inhibition of T cell proliferation by isolated Treg cells activated by CD3/CD28 T cell expander beads using a [³H]thymidine incorporation assay. The respective ratios of CD4⁺CD25⁺ conventional T cells: CD4⁺CD25⁺ regulatory T cells (Tcon:Treg) in each well are shown on the x-axis. Each well contained 5×10⁴ Tcon, with the exception of the ratios 0:1 (no Tcon) and 2:0 (1×10⁵ Tcon). Means (±SD) of [³H]thymidine incorporation in counts per minute (cpm) after 16 h are shown. Data from one of three representative experiments, each performed in triplicates are shown.

### EXAMPLE 1

### Abbreviations

CSFE: Carboxyfluorescein diacete succinimidyl-ester. EC: endothelial cells. HUVEC: human umbilical vein endothelial cells; HPMEC: human pulmonary microvascular endothelial cells. L1-CAM: L1 cell adhesion molecule; NRP-1: neuropilin-1; Tu PAMEC: tumour-derived pancreatic microvascular endothelial cells.

### Materials and Methods

### Tissue Samples

Pancreatic tissue samples from 24 patients with histologically confirmed primary pancreatic carcinomas were collected during primary tumour resection (pancreatectomy). Nonmalignant pancreatic tissue was also obtained during pancreatectomy and used as control tissue after pathologic exclusion of tumour infiltration. Tissue samples were either immediately processed or shock frozen in liquid nitrogen for immunohistology. Written informed consent was obtained from all participants and the protocol was approved by the Ethical Committee of the University of Heidelberg.

### Cells and Cell Culture

Microvascular endothelial cells were isolated from pancreatic tumour tissues. Tissues were washed in phosphate-buffered saline (PBS, Invitrogen, Karlsruhe, Germany), mechanically dissected into small pieces (approximately 1 mm²) and intensely resuspended with endothelial cell growth medium MV (ECGM) with supplement (5% fetal calf serum, 0.4% ECGS/H, 10 ng/mL epidermal growth factor, 1 μg/mL hydrocortisone, 1% penicillin/streptomycin; PromoCell, Heidelberg, Germany). Single cells were obtained from the suspension after filtering through 40 μm cell strainers (Falcon BD, Heidelberg, Germany) and washed with PBS. Endothelial cells (EC) were magnetically isolated using anti-CD31-Dynabeads (Dynal Biotech, Hamburg, Germany). Isolated EC were transferred into gelatin-coated (2%) cell culture flasks (TPP, Trasadingen, Switzerland) and cultured in supplemented ECBM until passage four. Human macrovascular umbilical vein endothelial cells (HUVEC) and human pulmonary microvascular endothelial cells (HMVEC-L; herein named HPMEC) (Provitro, Berlin, Germany) were cultured in endothelial cell growth medium MV (PromoCell, Heidelberg, Germany) until passage four. Panc1 cell line was cultured in Dulbecco's modified Eagle's medium supplemented with 10% fetal calf serum.

Cell lysates were generated by five cycles of freezing in liquid nitrogen and thawing and were then centrifuged (20 minutes at 30,000 g) to remove cell debris and organelles. Protein concentrations of the supernatants were determined by Bradford assay (BioRad, München, Germany). EC were always carefully washed with PBS to remove traces of respective cell lysates before use in subsequent experiments.

### Isolation of Treg Cell Subsets

Mononuclear cells were isolated from peripheral blood samples by density gradient centrifugation using Ficoll (Biochrom, Berlin, Germany). CD4⁺CD25⁺ Treg and CD4⁺CD25⁺ conventional T cells (Tcon) were then isolated using a CD4 CD25 regulatory T-cell isolation kit (Miltenyi, Bergisch Gladbach, Germany). The purity of isolated cells was 85-99%.

### Proliferation Assay

Tcon cells isolated from peripheral blood samples were incubated for 72 hours in 96-well plates (TPP, Trasadingen, Switzerland) alone or in co-culture with autologous Treg cells (always 5×10⁴ Tcon per well) at different ratios of Tcon:Treg (1:0, 2:1, 4:1, 8:1 and 16:1). In addition, 5×10⁴ Treg cells per well alone (0:1, negative control) and 1×10⁵ Tcon per well alone (2:0, positive control) were cultured separately under the same conditions. For activation of T cells, T cell cultures had been supplemented with CD3/CD28 T cell expander Dynabeads® (4 beads per cell, Invitrogen Karlsruhe, Germany). After 72 hours at 37° C., [³H]thymidine at 1 μCi per well was added for an additional 16 hours of culture. Proliferation of T cells was measured by determining the amount of incorporated [³H] using a scintillation counter (Liquid Scintillation Counter [1450 Micro Beta] Perkin Elmer, Wellesley, Mass.) in triplicate wells (n=3 independent experiments with 3 different donors).

### In Vitro Transmigration Assay

Transwell membranes (5 μm pore sizes; Costar, Corning, N.Y.) were coated for 60 minutes at 4° C. with 0.2% gelatine (Sigma-Aldrich, Munich, Germany). The medium in the lower chamber was supplemented with recombinant human stromal cell-derived factor 1β (rhSDF-1β, 100 ng/mL, PromoKine, Heidelberg, Germany) to establish a gradient for EC transmigration. EC were cultured with either tumour- or control-cell lysates (50 μg/mL) for three days, washed twice with PBS and added in the upper chamber (1×10⁵ per well). For blocking experiments 5-10 μg/mL mouse anti-human L1-CAM (L1-11A), mouse anti-human neuropilin-1 (Miltenyi Biotec, Bergisch Gladbach, Germany) or mouse IgG1 antibodies (as specificity control) (Santa Cruz Biotechnology, Heidelberg, Germany) were supplemented. Those cells that had transmigrated in the lower chamber were quantified 24 hours later by using a Casy® Cell Counter (Innovatis AG, Reutlingen, Germany). For transendothelial migration assay EC (2×10⁵ per well) were added on a gelatine coated membrane and cultured for two days on the membranes in supplemented ECGM until they reached confluency. EC monolayers were then washed twice with PBS and activated with recombinant human tumour necrosis factor-α (rhTNF-α, 400 U/mL, PromoKine, Heidelberg, Germany) for four hours. Tumour cells or T cells (1×10⁵ per well) were then added to the upper chamber. The medium in the lower chamber was supplemented with rhSDF-1β (100 ng/mL, PromoKine, Heidelberg, Germany), rhTGF-β1 (1-100 ng/ml, Promokine, Heidelberg) or 1×10⁴-1×10⁵ Tcon or Treg cells to establish a gradient for transendothelial cell transmigration. For activation of T cells, CD3/CD28 T cell expander Dynabeads® (4 beads per cell, Invitrogen Karlsruhe, Germany) were supplemented in the lower chamber. T cells, that had bound CD3/CD28 magnetic Dynabeads®, were magnetically separated from total cell suspension in the lower chamber immediately before cell counting and transmigrated tumour cells in the lower chamber were counted after 24 hours. For blocking experiments, tumour cells or EC were additionally incubated for four hours with the respective blocking monoclonal antibodies. All experiments were performed in triplicates (n=3 independent experiments).

### Flow Cytometry

Cells were washed with ice-cold PBS and detached with 5 mM EDTA/PBS. Single cell suspension of EC or isolated Treg cells (1.0×10⁵-1×10⁷ cells per well) were blocked with polyclonal human immunoglobulins (Endobulin, 2.5 mg/ml; Baxter Oncology, Frankfurt, Germany) and stained with the following antibodies: anti-human-CD3-PE-Cy5 (1:20, BD Pharmingen, Heidelberg, Germany), anti-human-CD4-FITC (1:10, BD Pharmingen, Germany), anti-human-CD25-PE (1:10, Miltenyi, Bergisch Gladbach, Germany), anti-human-FoxP3-APC (1:20, eBioscience, San Diego, Calif.), anti-human-neuropilin-1-PE (1:20, Miltenyi Biotec, Bergisch Gladbach, Germany) or anti-human-L1-CAM (1 mg/ml, 1:50, L1-11A) in PBS/3% FCS for 30 min on ice. L1-CAM antibody was detected by a goat anti-mouse phycoerythrine secondary antibody (1:400, Dianova, Hamburg, Germany). Dead cells, which were labeled with 1 μg/ml propidium iodide (Abeam, Cambridge, UK) immediately before flow cytometry, were excluded from analysis. Recordings were made from at least 1×10⁵ cells on a FACSCanto II flow cytometer (Becton Dickinson, Heidelberg, Germany) and analyzed using FlowJo 6.4 software (TreeStar, San Carlos, Calif.).

### Immunohistochemistry

Pieces of freshly isolated tumour and control tissues were embedded in Tissue Tek embedding medium, snap frozen in liquid nitrogen, and stored at −80° C. until use. Cryosections (5 μm) were prepared from frozen tissue, fixed in ice-cold acetone, blocked with 4% goat serum (Invitrogen, Karlsruhe, Germany) and incubated with the following primary antibodies: mouse anti-human-L1-CAM (1 mg/ml, 1:50, L1-11A), rabbit anti-human-CD31 (1:50, Spring bioscience, Freemont, Calif.) followed by detection with the following secondary antibodies: goat-anti-mouse-Cy3 (red; Dianova, Hamburg, Germany) and goat-anti-rabbit-AlexaFluor-488 (green; Invitrogen, Karlsruhe, Germany) (all diluted 1:500). Slides were washed three times with PBS and 4′,6-diamidino-2-phenylindol (DAPI staining solution, Hoechst, Darmstadt, Germany) was added in a dilution of 1:3000 to detect nuclei. After antibody staining, tissue auto-fluorescence was blocked with CuSO₄ solution (1-10 mM CuSO₄ in 50 mM ammonium acetate buffer, pH 5.0; Sigma, Deisenhofen, Germany) and slides were covered with glycerin-gelatin (Merck, Darmstadt, Germany). Slides were evaluated by automatic determination of stained areas using AnalySIS Software® (Olympus Soft Imaging Solutions, Muenster, Germany). Quantitative analysis of slides was always based on a minimum of triplicate sections per sample (n=20 different donors).

### Immunocytochemistry

EC were added on fibronectin (100 μg/mL) coated Lab-Tek™ chamber slides (Nunc, Wiesbaden, Germany) and cultured in supplemented ECGM until they reached confluence. For activation, EC were stimulated with different cell lysates (50 μg/mL) for three days or rhTNF-α (200 U-400 U/mL) for 24 hours, respectively. Cells were fixed with 4% paraformaldehyde (Merck, Darmstadt, Germany), permeabilized with 0.1% Triton-X 100 (AppliChem, Darmstadt, Germany) and stained with anti-human-L1-CAM (1 mg/ml, 1:50, L1-11A) followed by goat anti-mouse-Cy3 (1:500, Firma) secondary antibody. Slides were evaluated by counting labeled cells (n=3 independent experiments).

### In Vitro Tube Formation Assay

EC (2×10⁴ per well) were added on 96-well plate coated with 100 μl matrigel (BD Pharmingen, Heidelberg, Germany) and incubated for 24 hours. For blocking experiments, 10 μg/mL anti-human-L1-CAM (L1-11A), anti-human-neuropilin-1 (Miltenyi Biotec, Bergisch Gladbach, Germany) or mouse IgG1 antibodies (as specificity control) (Santa Cruz Biotechnology, Heidelberg, Germany) were supplemented. Tube formation was quantified by counting the number of vascular joints in 2 non-overlapping fields (each field defined as the area visualized by a 10× magnification lens). All experiments were performed in triplicates (n=3 independent experiments).

### In Vitro Adhesion Assay

EC were seeded on fibronectin (100 μg/mL) coated Lab-Tek™ chamber slides (Nunc, Wiesbaden, Germany) and cultured in supplemented ECGM until they reached confluency. EC were activated with rhTNF-α (400 U/mL) for four hours and endothelial and tumour cells were preincubated with inhibiting antibodies for four hours, respectively. For quantification of tumour cell adhesion capacity tumour cells were labelled with 25 μM carboxyfluorescein diacetate succinimidyl ester (CFSE, Invitrogen, Karlsruhe, Germany) and added to the EC monolayer for 60 min. Non-adherend tumour cells were removed by three washing steps with PBS and slides were covered with glycerin-gelatin (Merck, Darmstadt, Germany). Tumour cell adhesion was evaluated by counting 5 non-overlapping fields (each field defined as the area visualized by a 10× magnification lens). All experiment were performed in triplicates (n=3 independent experiments).

### Quantitative RT-PCR

mRNA was isolated using High Pure RNA Isolation Kit according to the manufacturer's protocol (Roche Diagnostics, Mannheim, Germany). mRNA was processed for cDNA synthesis using Superscript II reverse transcriptase (RT) with oligo(dT) (Invitrogen). cDNA was purified on Microspin G-50 columns (Amersham Biosciences, Freiburg, Germany) and quantitated by NanoDrop spectrophotometer (ND-1000, Kisker-Biotechnology, Steinfurt, Germany). Primers were designed with the DNA Star Program. Amplification reactions were analyzed on ABI 7300 sequence detection system using SYBR Green mastermix (Applied Biosystems, Foster City, Calif.) Biosystems, Darmstadt, Germany). β-Actin was used as an internal standard. Sequence of primers is available on request.

### Statistical Analysis

P values were calculated by using two-sided student t test. A P-value lower than 0.05 was considered statistically significant.

### EXAMPLE 2

### Enhanced Endothelial L1-CAM Expression in Pancreatic Carcinoma Tissue

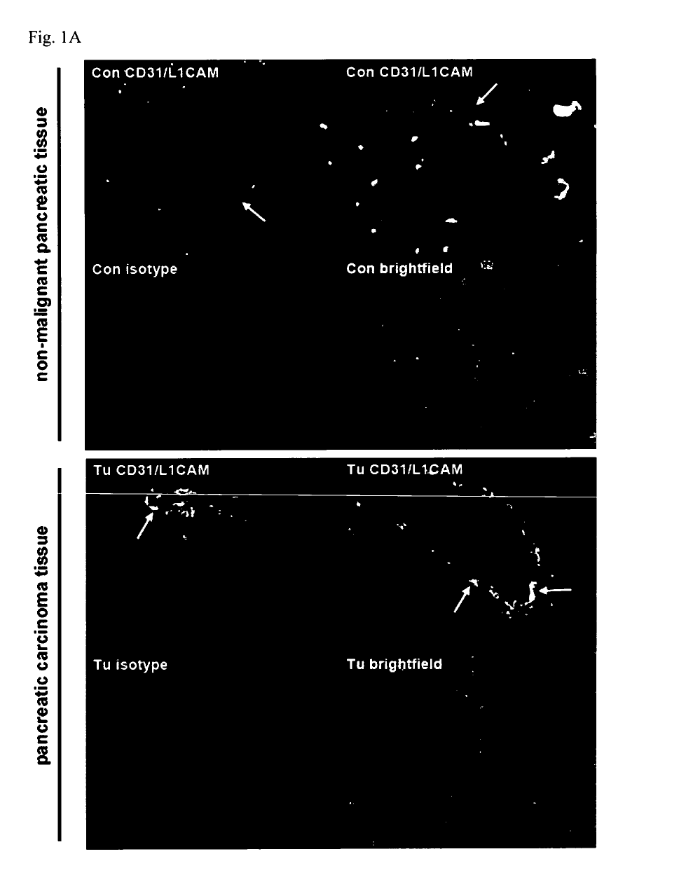

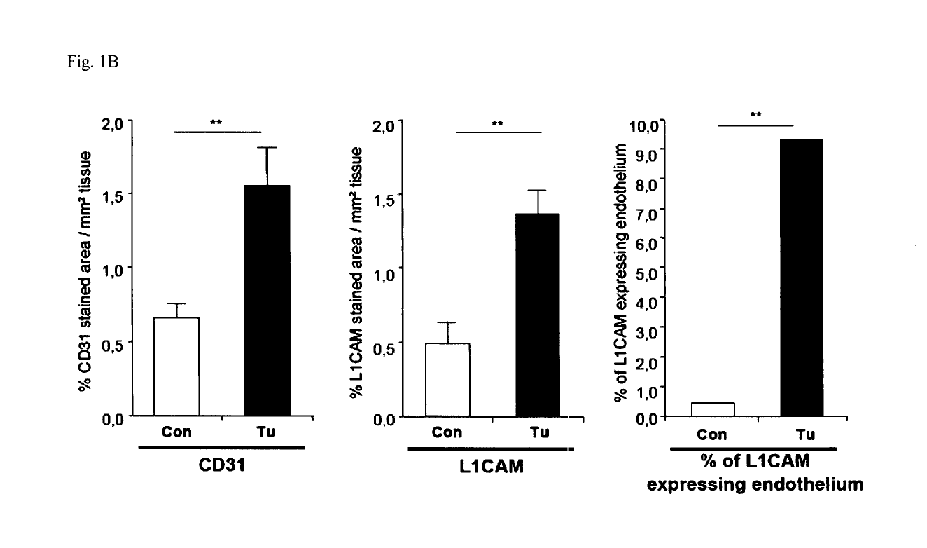

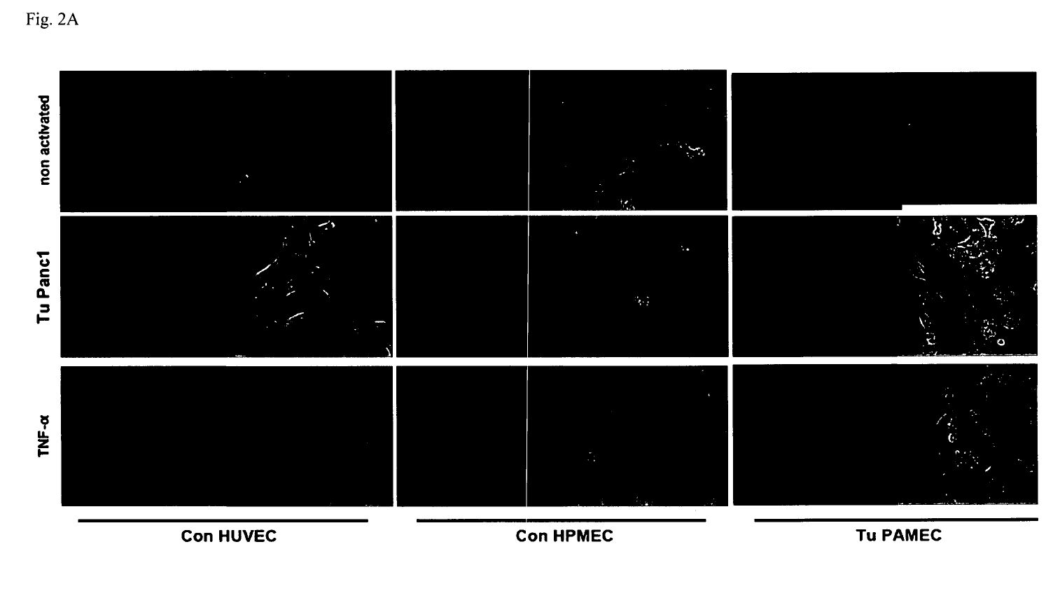

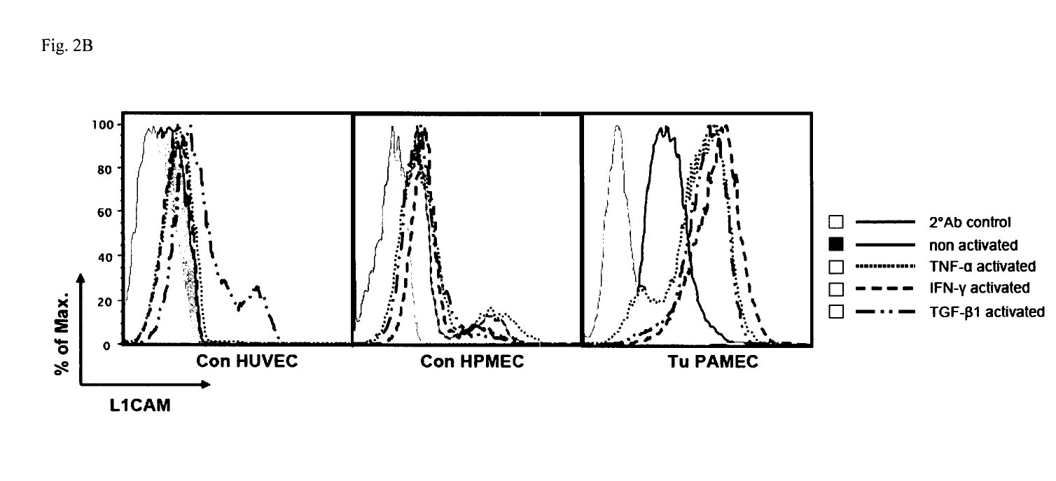

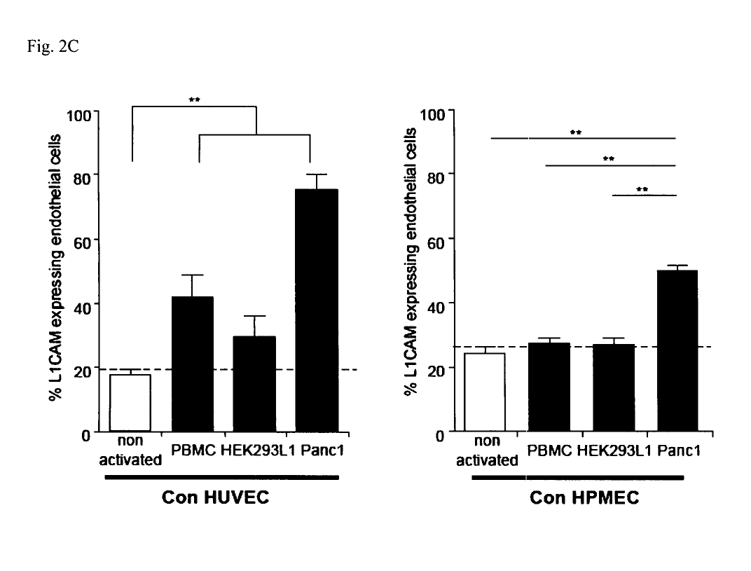

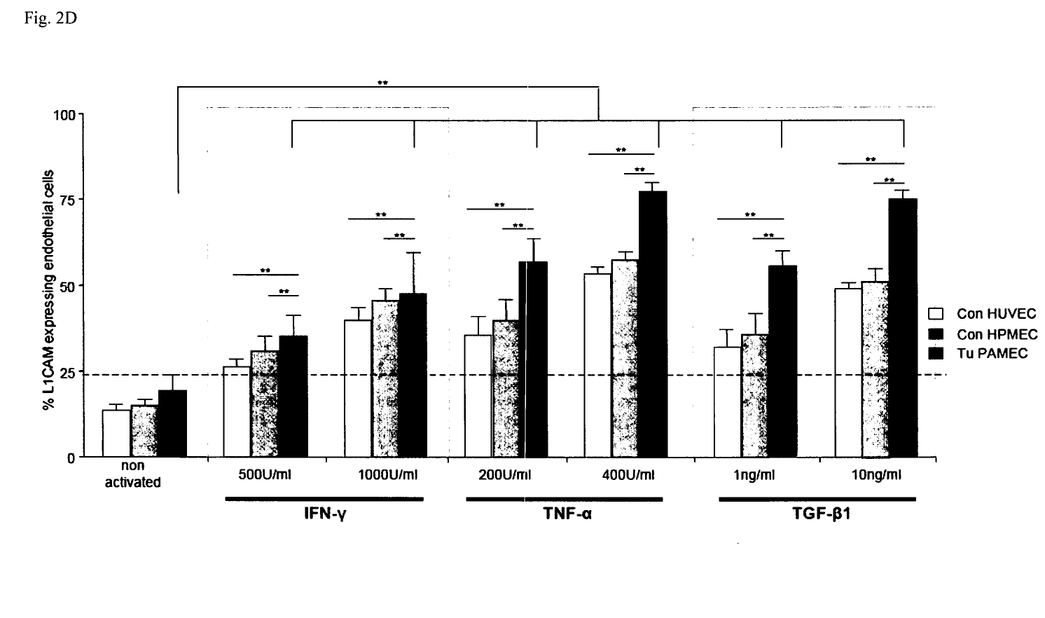

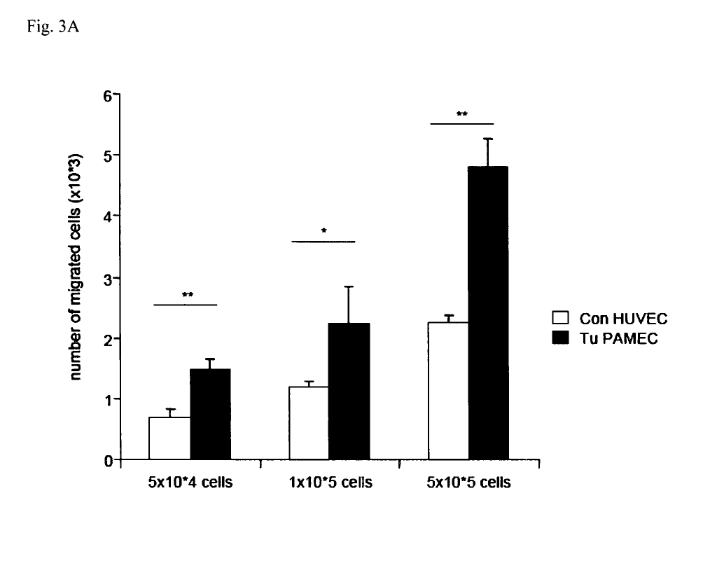

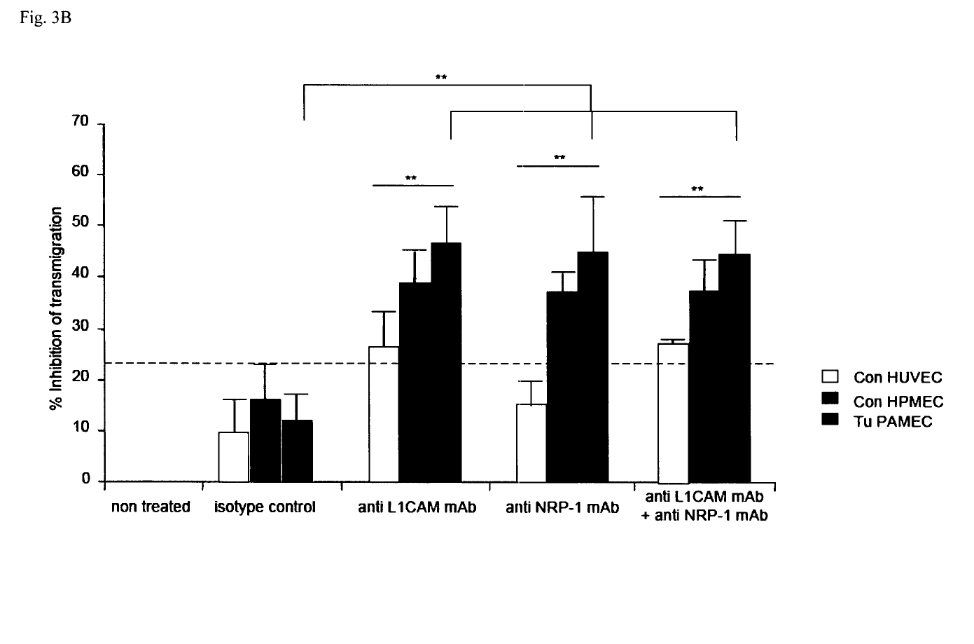

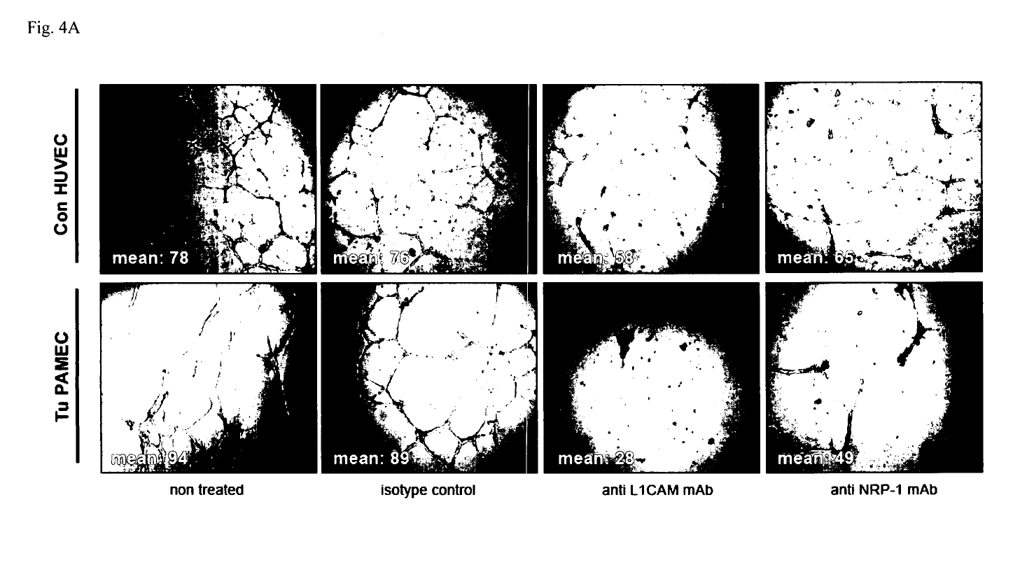

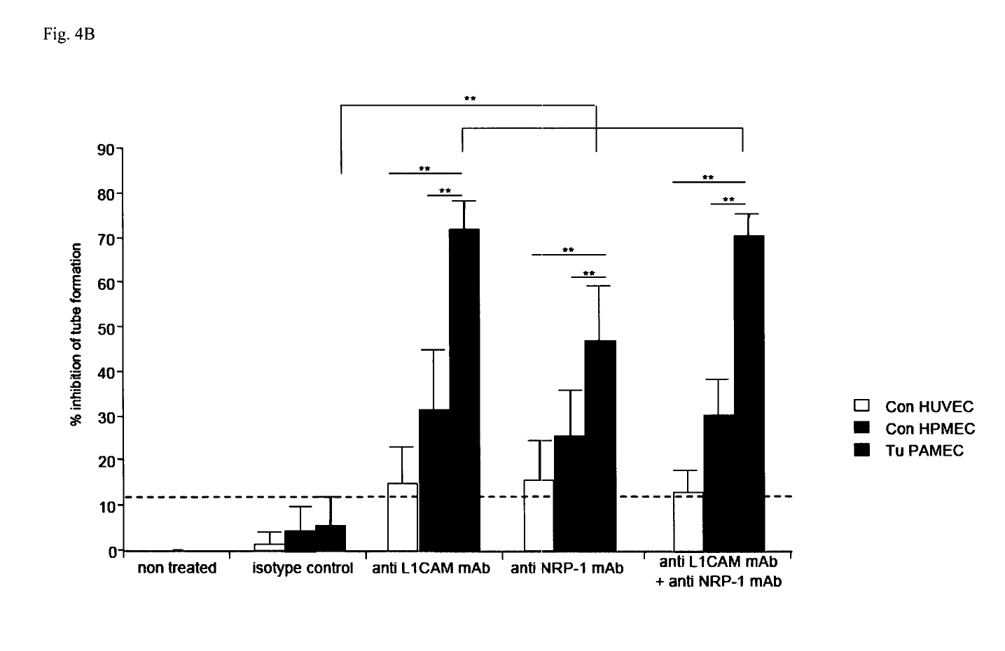

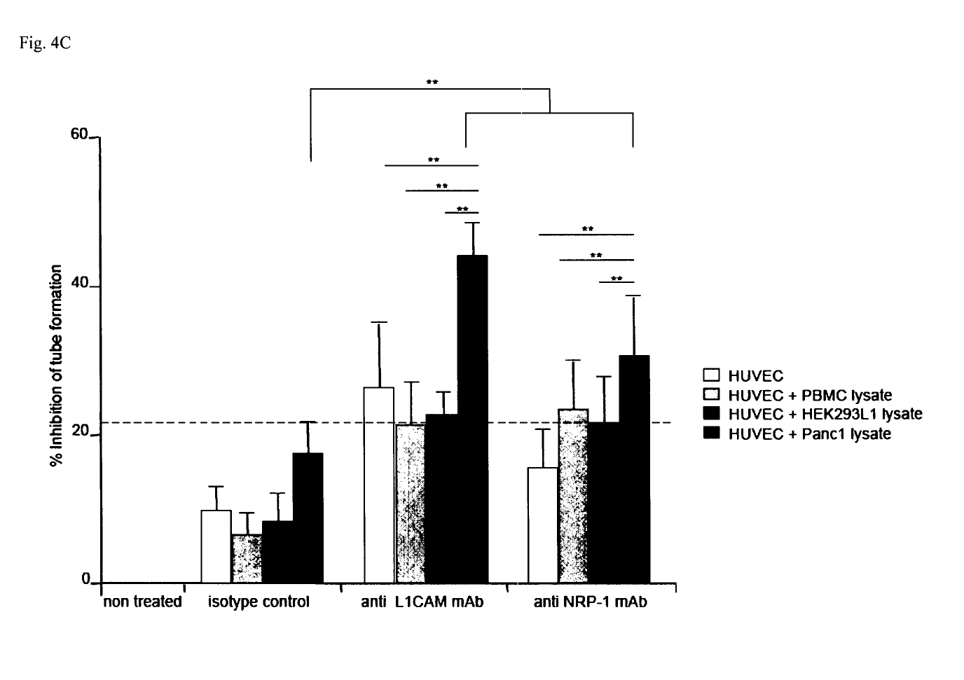

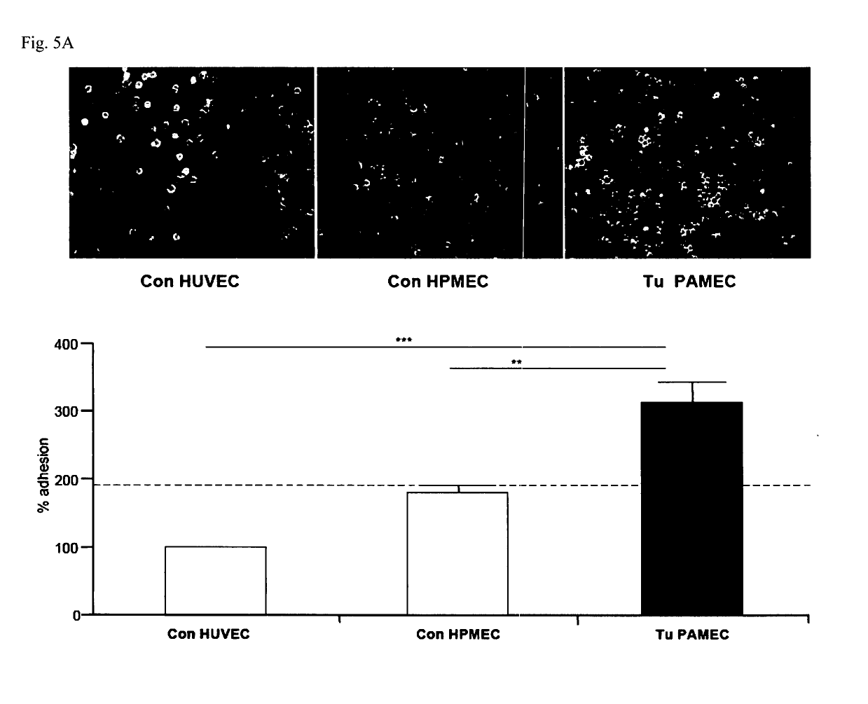

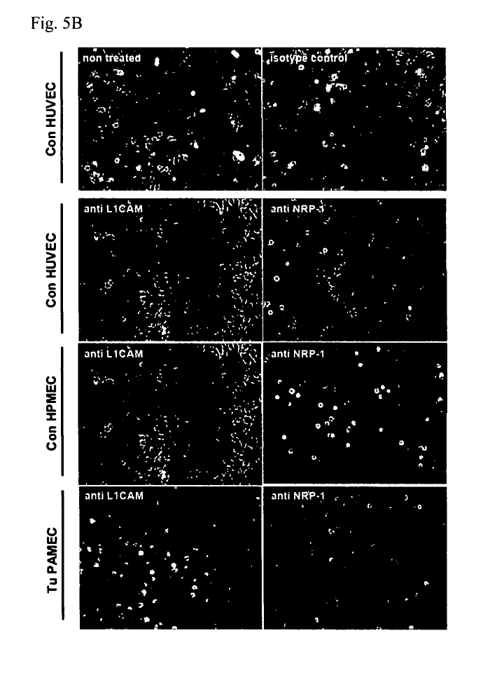

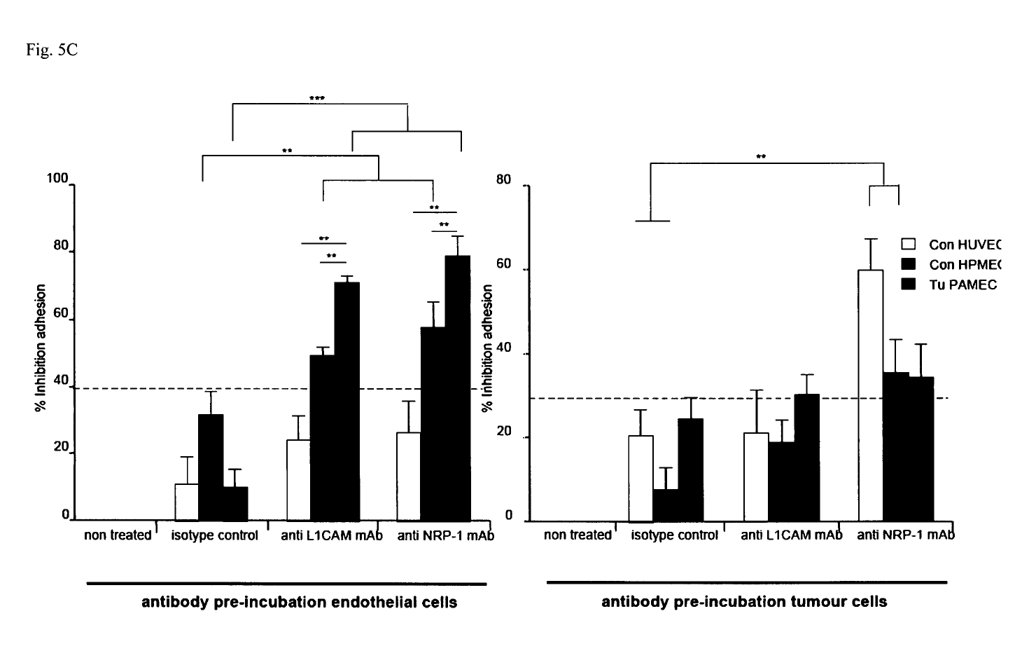

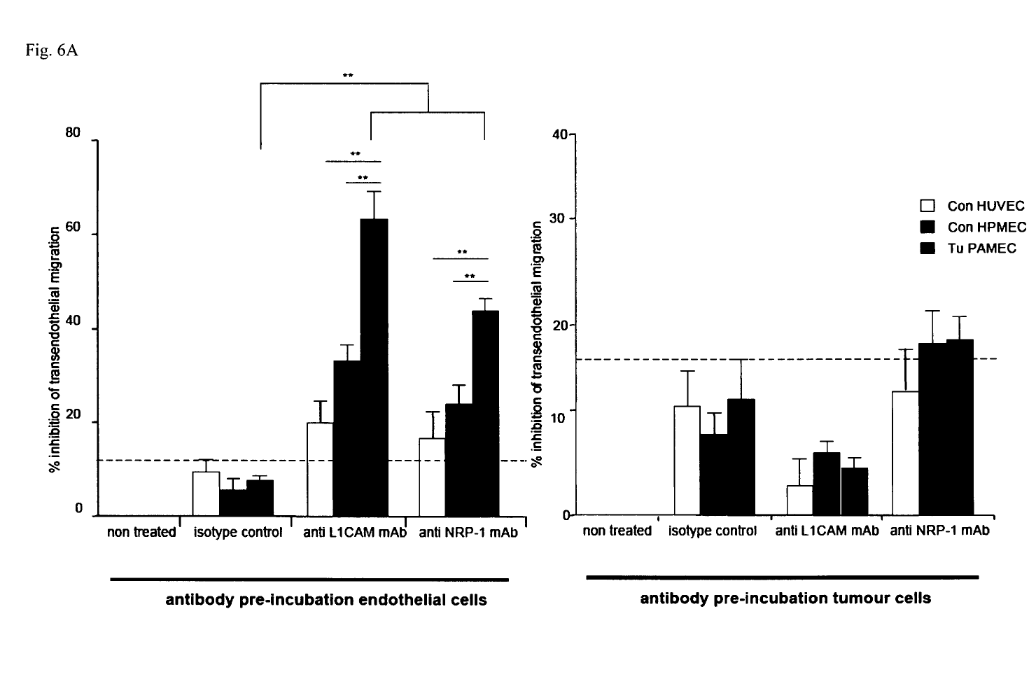

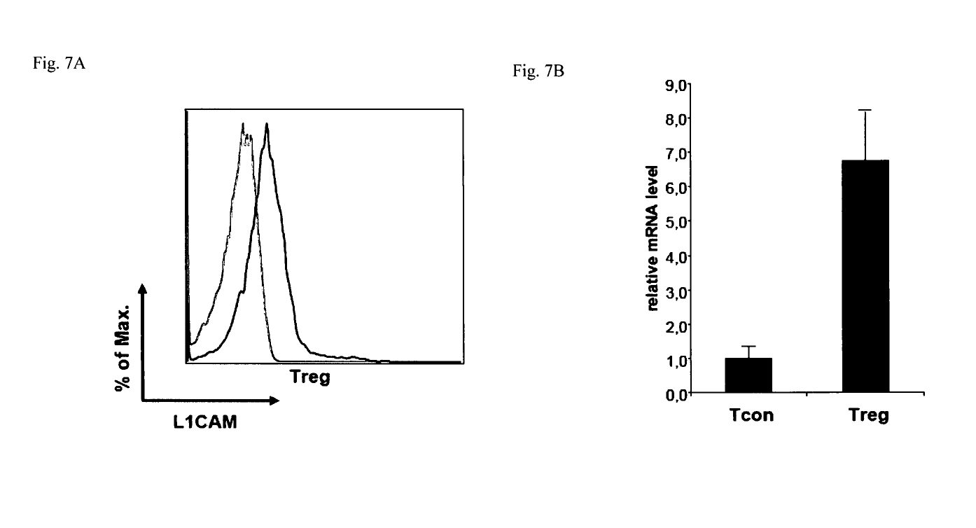

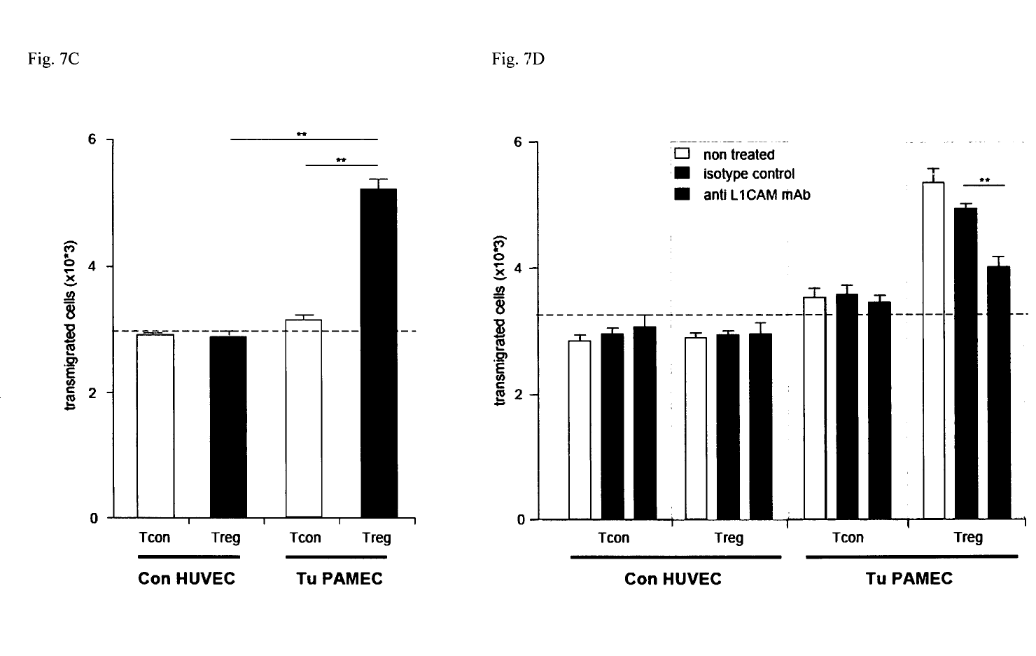

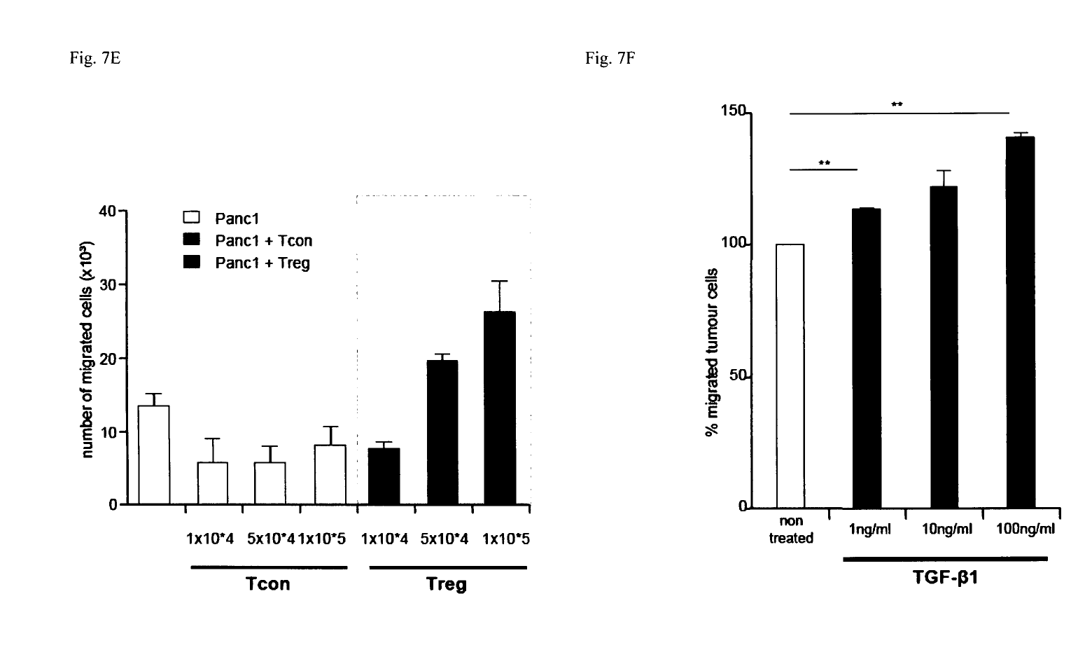

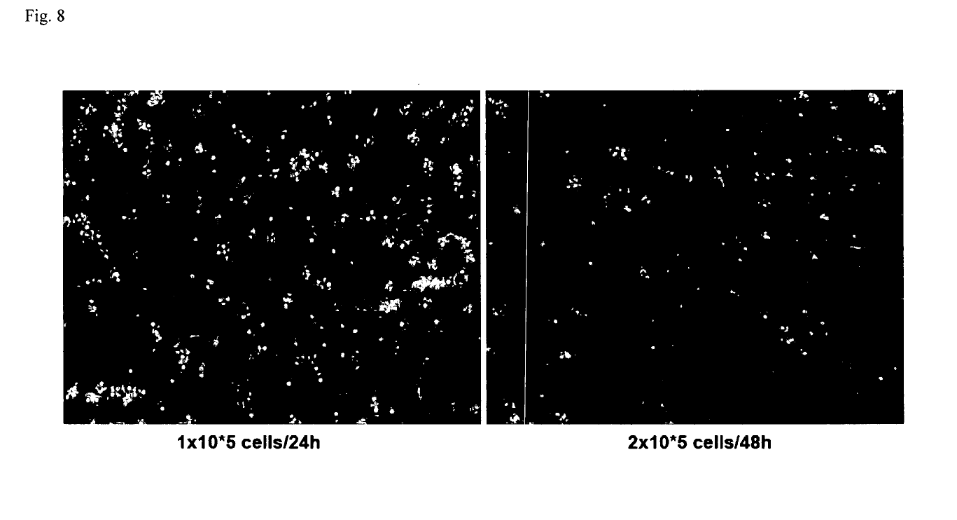

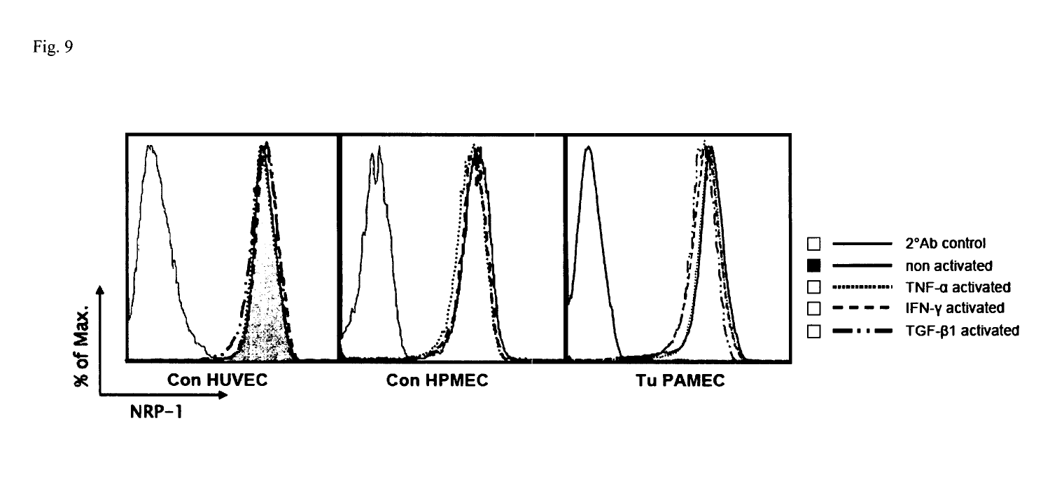

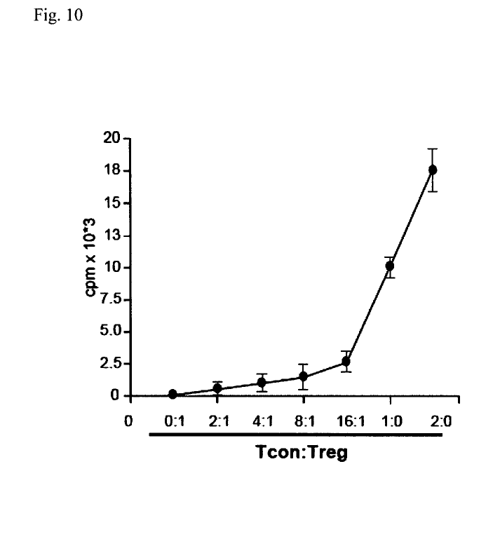

We analysed L1-CAM expression in tumour infiltrating and peritumoral vessels of pancreatic carcinoma in comparison to non-malignant pancreatic tissue samples using immunohistochemical staining. For this the pancreatic tumour tissue (Tu) or corresponding non-malignant pancreatic tissue (Con) were stained with monoclonal antibodies to L1-CAM (red) and CD31 (green) (FIG. 1A). L1-CAM showed enhanced expression in pancreatic carcinoma tissues (n=24) compared to non-malignant pancreatic tissue (n=20) (FIG. 1B). The enhanced L1-CAM expression correlated with increased vascularization of the pancreatic tumour tissue (FIG. 1B; 0.7% vs. 1.6%; p=0.001). The tumour associated endothelium showed enhanced L1-CAM expression compared to endothelium of non-malignant pancreatic tissue (FIG. 1B; 0.4% vs. 9.3%).

To demonstrate that the staining detected L1-CAM in a full-length form and not as a soluble molecule (devoid of the cytoplasmic tail) we used the mAb 74-5H7 to the cytoplasmic portion. The detection of the cytoplasmic portion of endothelial L1-CAM in non-malignant and tumour tissue confirmed the endogenous expression of the full-length molecule (data not shown).

### EXAMPLE 3

### Imitation of Tumour Micromilieu Augments L1-CAM Expression of Endothelial Cells In Vitro

We examined whether tumour derived factors might enhance L1-CAM expression of EC. To address this question in vitro, we analyzed non-malignant macrovascular HUVEC (Con HUVEC) and microvascular HPMEC (Con HPMEC) and tumour-derived pancreatic microvascular endothelial cells (Tu PAMEC) isolated from pancreatic tumour tissues (Nummer et al., J Natl Cancer Inst 2007; 99:1188-1199). For imitation of the tumour micromilieu we used Panc1 tumour cell lysate or PBMC and HEK293-L1 non-tumour cell lysates. Incubation for 72 h of non-malignant and tumour-derived EC with Panc1 tumour cell lysate increased L1-CAM expression of EC in vitro (FIGS. 2A and C). In contrast, the incubation with control lysates did not show an increase of L1-CAM expression (FIGS. 2A and C). As HEK293-L1 and Panc1 cells are L1-CAM positive but upregulation was only seen with Panc1 lysate, these results argue against a passive uptake of soluble L1-CAM from the cell lysate.

The effect of Panc1 tumour cell lysate on L1-CAM expression could be mimicked by treatment of EC with proinflammatory cytokines such as tumour necrosis factor-α (TNF-α), interferon-γ (IFN-γ) or tumour growth factor-131 (TGF-β1). Significantly enhanced L1-CAM expression was observed after TNF-α, IFN-γ or TGF-β1 stimulation (FIGS. 2B and D). Non-activated Tu PAMEC already showed a higher expression level of L1-CAM than non-malignant EC (Con HUVEC and Con HPMEC). Activation with TNF-α, IFN-γ or TGF-β1 further enhanced L1-CAM expression (FIGS. 2B and 2D).

We also evaluated the expression level of the L1-CAM ligand NRP-1 on EC. All three EC lineages showed high NRP-1 expression levels that were not further increased by stimulation with TNF-α, IFN-γ or TGF-β1 for 24 h (FIG. 9).

### EXAMPLE 4

### L1-CAM Unfolds Tumour Endothelial Cell Selective Angiogenic and Migratory Capacities In Vitro

As L1-CAM over-expression in tumour cells augments the haptotactic motility on extracellular matrix proteins (Mechtersheimer et al., 2001; Silletti et al., 2004), we evaluated whether its expression in EC could affect the migratory capacity using an in vitro SDF-1β stimulated transmigration assays. Analysis of the basal migratory capacity was first investigated without SDF-1β stimulation. The results revealed a significantly higher transmigratory capacity of Tu PAMEC than non-malignant EC in a cell-dose dependent manner (FIG. 3A). Inhibition of transmigration by specific mAb to L1-CAM or NRP-1, respectively, showed Tu PAMEC specific inhibition of transmigration in comparison to non-malignant EC or isotype control, respectively (FIG. 3B). Inhibition of transmigration in presence of both mAb did not enhance the blocking capacity of individual mAbs (FIG. 3B).

To evaluate the putative relevance of L1-CAM in the angiogenic outgrowth of the tumour vasculature, we performed tube formation assays in vitro. Quantification of tube structures demonstrated enhanced formation of tubes of Tu PAMEC in comparison to non-malignant EC (FIG. 4A). Blocking with anti L1-CAM or anti-NRP-1 mAbs showed enhanced inhibition of Tu PAMEC tube formation in comparison to non-malignant EC and respective isotype control (FIG. 4B) correlating with the amount of L1-CAM expression. Again, inhibition in the presence of both mAb was not additive.

Incubation of Con HUVEC with non tumour lysates (PBMC and HEK293-L1) did not significantly enhance the inhibition of tube formation in comparison to non-treated HUVEC (FIG. 4C).

### EXAMPLE 5

### Tumour Endothelial Cell Expression of L1-CAM Supports Cell Adhesion and Transmigration of Carcinoma Cells In Vitro

Previous work has shown that L1-CAM is involved in the transmigration of melanoma cells through monolayers of EC (Voura et al., 2001). To investigate a putative role of L1-CAM for the metastatic spread of pancreatic carcinoma, we performed in vitro cell adhesion assays using Panc1 cells. CSFE-labeled Panc1 cells were incubated for 60 min on TNFα pre-stimulated EC monolayers. Quantification of adhering cells showed increased adhesion of Panc1 tumour cells to Tu PAMECs in comparison to non-malignant EC monolayers (FIG. 5A). Blocking with anti L1-CAM or anti-NRP-1 mAbs showed enhanced inhibition of Panc1 tumour cell adhesion to Tu PAMEC monolayer in comparison to non-malignant EC monolayer and respective isotype control (FIG. 5B). Interestingly, pre-incubation with L1-CAM or NRP-1 mAbs of EC showed enhanced inhibition of Panc1 tumour cell adhesion to Tu PAMEC compared to non-malignant EC (FIG. 5C). Pre-incubation of Panc1 tumour cells with L1-CAM mAb followed by the removal of mAb did not significantly inhibit Panc1 tumour cell adhesion to EC monolayers. However, pre-incubation of Panc1 tumour cells with respective NRP-1 mAb showed increased inhibition of Panc1 tumour cell adhesion to non-malignant EC monolayer suggesting that L1-CAM on EC interacted preferentially with NRP-1 on carcinoma cells.

To allow the analysis of transmigration through EC monolayers, we first established conditions to achieve a close EC monolayer on the membrane of the transmigration chamber. A close EC monolayer was obtained by seeding 2×10⁵ cells for a growing period of 48 h (FIG. 8). These conditions were used for all transendothelial migration assays. Evaluation of transendothelial migration capacity of Panc1 tumour cells demonstrated increased transendothelial migration through Tu PAMEC in comparison to non-malignant EC monolayer (FIG. 6, left panel). Additionally, pre-incubation of EC with L1-CAM or NRP-1 mAbs showed enhanced inhibition of transendothelial migration through Tu PAMEC compared to non-malignant EC monolayer (FIG. 6, left panel). In contrast, pre-incubation with L1-CAM or NRP-1 mAbs of Panc1 tumour cells did not inhibit transendothelial migration (FIG. 6, right panel).

### EXAMPLE 6

### Tumour Endothelial Cell Expression of L1-CAM Supports Transendothelial Migration of Regulatory T Cells In Vitro

EC represent a barrier for blood-borne tumour cells but also for lymphocytes. Recent work has revealed that pancreatic tumours can alter the expression of adhesion molecules on the surface of EC to allow selective transmigration of regulatory T cells (Treg) from peripheral blood to the tumour site (Nummer et al., 2007).

Evaluation of L1-CAM expression of isolated Treg cells was done using flow cytometry analysis. Enhanced L1-CAM expression could be shown on CD3⁺, CD4⁺, CD25⁺ and FoxP3⁺ gated Treg cells (FIG. 7A) which could be confirmed by quantitative RT-PCR analysis (FIG. 7B). To assess whether cells with a Treg phenotype also displayed functional Treg cell characteristics, we tested their capacity to suppress the proliferation of autologous cocultured CD4⁺CD25⁺ conventional T cells (Tcon) after activation with CD3/CD28 expander beads. Blood-derived Treg cells efficiently suppressed Tcon cell proliferation in a dose-dependent manner (FIG. 10).

Analysis of the transendothelial migration capacity of Treg cells demonstrated enhanced migration through Tu PAMEC monolayer in comparison to Tcon cells. Transendothelial migration of Treg cells showed also increased transendothelial migratory capacity through Tu PAMEC monolayer in comparison to non-malignant EC monolayer (FIG. 7C). Blocking with mAb to L1-CAM showed a Treg specific inhibition of transendothelial migration in comparison to Tcon or isotype control, respectively (FIG. 7D). Transendothelial migration through non-malignant EC monolayer was not inhibited in the presence of L1-CAM mAb (FIG. 7D).

Previous work has demonstrated that Tregs can infiltrate pancreatic carcinoma tissues (Nummer et al., 2007). To investigate a possible impact of Tregs on transendothelial migration capacity of tumour cells, we evaluated the influence of soluble factors released of Treg cells on transendothelial migration. Instead of chemokine stimulus we used activated Tcon or Treg cells, respectively, for induction of Panc1 transendothelial migration. Quantification of transmigrated Panc1 tumour cells conditioned by Treg cells showed enhanced transendothelial migration in a cell-dose dependent manner. In contrast transendothelial migration of Panc1 tumour cells was inhibited by using Tcon cells as stimulus (FIG. 7E). As TGFβ1 is one of the released proinflammatory cytokines by Tregs, we used TGFβ1 to stimulate Panc1 transendothelial migration. Evaluation of Panc1 transendothelial migration showed increased migration in a dose-dependent manner (FIG. 7F). However, TFGβ1 stimulated migration of Panc1 cells through gelatine-coated membranes did not increase the migratory capacity of Panc1 tumour cells (data not shown).

### Discussion of the Examples

Here we demonstrate that tumour endothelium of pancreatic carcinoma, but not endothelium in non-malignant pancreatic tissue of the same patients, showed increased L1-CAM expression in situ. This was confirmed in isolated Tu PAMEC kept in short term culture in vitro. We observed a significantly higher L1-CAM expression level on Tu PAMEC compared to non-malignant EC cells. We observed that L1-CAM expression could be augmented by incubation of non-malignant EC (Con HUVEC and Con HPMEC) with Panc1 tumour cell lysates or proinflammatory cytokines such as TNF-α, IFN-γ or TGF-β1. Antibodies to L1-CAM and NRP-1 blocked tube formation and transmigration of tumour-derived EC in vitro. Enhanced L1-CAM expression increased cell adhesion of Panc1 cells and Treg cells to EC monolayer and supported transendothelial migration. Our data demonstrate an unrecognized role for L1-CAM selectively on tumour endothelium that could be important for the angiogenic and metastasizing processes during tumour progression.

### REFERENCES

Adamis A P, Aiello L P, D'Amato R A. Angiogenesis and ophthalmic disease. Angiogenesis. 1999; 3: 9-14
Arlt et al., M J, Novak-Hofer I, Gast D, Gschwend V, Moldenhauer G, Grunberg J, et al. Efficient inhibition of intra-peritoneal tumor growth and dissemination of human ovarian carcinoma cells in nude mice by anti L1-cell adhesion molecule monoclonal antibody treatment. Cancer Res 2006; 66:936-43
Blood C. H., and Zetter B. R. Tumor interactions with the vasculature: angiogenesis and tumor metastasis. Biochim Biophys Acta. 1990; 1032(1):89-118
Brummendorf T, Kenwrick S, Rathjen F G. Neural cell recognition molecule L1: from cell biology to human hereditary brain malformations. Curr Opin Neurobiol. 1998; 8:87-97.
Carmeliet P. Angiogenesis in life, disease and medicine. Nature. 2005; 438: 932-6.
Castellani V, De Angelis E, Kenwrick S, Rougon G. Cis and trans interactions of L1 with neuropilin-1 control axonal responses to semaphorin 3A. Embo J. 2002; 21:6348-6357.
Costa C, Incio J, Soares R. Angiogenesis and chronic inflammation: cause or consequence? Angiogenesis. 2007; 10:149-66
Ebeling O, Duczmal A, Aigner S, et al. L1 adhesion molecule on human lymphocytes and monocytes: expression and involvement in binding to alpha v beta 3 integrin. Eur J. Immunol. 1996; 26:2508-2516.
Elbashir S. M., Lendeckel W., and Tuschl T. RNA interference is mediated by 21- and 22-nucleotide RNAs. Genes Dev 2001; 15:188-200.
Eichmann A, Makinen T, Alitalo K. Neural guidance molecules regulate vascular remodeling and vessel navigation. Genes Dev. 2005; 19: 1013-1021.
Felding-Habermann B, Silletti S, Mei F, et al. A single immunoglobulin-like domain of the human neural cell adhesion molecule L1 supports adhesion by multiple vascular and platelet integrins. J. Cell Biol. 1997; 139:1567-1581.
Ferrara N, and Kerbel R S. Angiogenesis as a therapeutic target. Nature. 2005; 438: 967-74.
Folkman J. and Klagsbrun M. Angiogenic factors. Science 1987; 235: 442-7.
Folkman J., Watson K., Ingber D, Hanahan D. Induction of angiogenesis during the transition from hyperplasia to neoplasia. Nature 1989; 339: 58-61.
Folkman J., The role of angiogenesis in tumor growth. Semin Cancer Biol. 1992; 3: 65-71.
Folkman J., Angiogenesis in cancer, vascular, rheumatoid and other disease. Nat Med 1995; 1:27-31.
Fogel M, Gutwein P, Mechtersheimer S, Riedle S, Stoeck A, Smirnov A, et al. L1 expression as a predictor of progression and survival in patients with uterine and ovarian carcinomas. Lancet 2003; 362:869-75.
Gast D, Riedle S, Issa Y, Pfeifer M, Beckhove P, Sanderson M P, Arlt M, Moldenhauer G, Fogel M, Krüger A, and Altevogt P. The cytoplasmic part of L1-CAM controls growth and gene expression in human tumors that is reversed by therapeutic antibodies. Oncogene 2008; 27:1281-1289.
Huszar M, Moldenhauer G, Gschwend V, Ben-Arie A, Altevogt P, Fogel M: Expression profile analysis in multiple human tumors identifies L1 (CD171) as a molecular marker for differential diagnosis and targeted therapy. Hum Pathol 37:1000-1008, 2006
Kaifi J T, Reichelt U, Quaas A, et al. L1 is associated with micrometastatic spread and poor outcome in colorectal cancer. Mod Pathol. 2007; 20:1183-1190.
Mechtersheimer S, Gutwein P, Agmon L N, et al. Ectodomain shedding of L1 adhesion molecule promotes cell migration by autocrine binding to integrins. J. Cell Biol. 2001; 155:661-674.
Meli M L, Carrel F, Waibel R, Amstutz H, Crompton N, Jaussi R, Moch H, Schubiger P A, Novak-Hofer I. Anti-neuroblastoma antibody chCE7 binds to an isoform of L1-CAM present in renal carcinoma cells. Int J Cancer, 1999; 83: 401-408
Moos M, Tacke R, Scherer H, Teplow D, Fruh K, Schachner M. Neural adhesion molecule L1 as a member of the immunoglobulin superfamily with binding domains similar to fibronectin. Nature. 1988; 334:701-703.
Mujoo K., Spiro R. C., Reisfeld R. A. Characterization of a unique glycoprotein antigen expressed on the surface of a human neuroblastoma cells. J. Biol. Chem. 1986; 261; 10299-305).
Neufeld G, Cohen T, Shraga N, Lange T, Kessler O, Herzog Y. The neuropilins: multifunctional semaphorin and VEGF receptors that modulate axon guidance and angiogenesis. Trends Cardiovasc Med. 2002; 12:13-19.
Nitecki S S, San M G, Colby T V, van Heerden J A. Long-term survival after resection for ductal adenocarcinoma of the pancreas. Is it really improving? Ann Surg. 1995; 221:59-66.
Novak-Hofer I., Cohrs S., Grünberg J., Friedl A., Schlatter M. C., Pfeiffer M., Altevogt P., and Schubiger P. A. Antibodies directed against L1-CAM synergize with Genistein in inhibiting growth and survival in SKOVip human ovarian cancer cells. Cancer Lett. 2008; 261; 193-204.
Nummer D, Suri-Payer E, Schmitz-Winnenthal H, et al. Role of tumor endothelium in CD4+CD25+ regulatory T cell infiltration of human pancreatic carcinoma. J Natl Cancer Inst. 2007; 99:1188-1199.
Oleszewski, M., Gutwein, P., von der Lieth, W., Rauch, U., Altevogt, P. Characterization of the L1-neurocan binding site. Implications for L1-L1 homophilic binding. J. Biol. Chem. 2000; 275: 34478-34485.
Patel K, Kiely F, Phimister E, Melino G, Rathjen F, Kemshead J T: The 200/220 kDa antigen recognized by monoclonal antibody (MAb) UJ127.11 on neural tissues and tumors is the human L1 adhesion molecule. Hybridoma 1991; 10:481-491.
Patel K, Banting G, Frost G, Kemshead J T. X-linked gene MIC5 codes for the L1 adhesion molecule recognized by monoclonal antibody R1. Cancer Genet Cytogenet. 1992; 60:20-22.
Perez-Atayde A R, Sallan S E, Tedrow U, Connors S, Allred E, Folkman J. Spectrum of tumor angiogenesis in the bone marrow of children with acute lymphoblastic leukemia. Am J Pathol. 1997; 150:815-21
Schachner M. Neural recognition molecules and synaptic plasticity. Curr Opin Cell Biol 1997; 9:627-34
Sebens Müerköster S, Werbing V, Sipos B, et al. Drug-induced expression of the cellular adhesion molecule L1-CAM confers anti-apoptotic protection and chemoresistance in pancreatic ductal adenocarcinoma cells. Oncogene. 2007; 26:2759-2768.
Silletti S, Yebra M, Perez B, Cirulli V, McMahon M, Montgomery A M. Extracellular signal-regulated kinase (ERK)-dependent gene expression contributes to L1 cell adhesion molecule-dependent motility and invasion. J Biol. Chem. 2004; 279:28880-28888.
Thies A, Schachner M, Moll I, et al. Overexpression of the cell adhesion molecule L1 is associated with metastasis in cutaneous malignant melanoma. Eur J Cancer. 2002; 38:1708-1716.
Voura E B, Ramjeesingh R A, Montgomery A M, Siu C H. Involvement of integrin alpha(v)beta(3) and cell adhesion molecule L1 in transendothelial migration of melanoma cells. Mol Biol Cell. 2001; 12:2699-2710.
Wolff J M, Frank R, Mujoo K, Spiro R C, Reisfeld R A, Rathjen F G: A human brain glycoprotein related to the mouse cell adhesion molecule L1. J Biol Chem 263:11943-11947, 1988.

1.-30. (canceled)

31. A method for the treatment of a disease in a patient which can be treated by inhibition of angiogenesis, wherein an effective amount of anti L1-CAM antibody is administered to said patient and wherein the administration of said anti L1-CAM antibody results in the inhibition of angiogenesis.

32. The method of claim 31, wherein the effective amount of L1-CAM antibody is in a dosage range of about 20-500 μg/kg body weight for intravenous administration, or in a dosage range of about 0.01 pg/kg to 1 mg/kg body weight for intranasal administration.

33. The method of claim 31, wherein the disease is characterized by
a.) a pathological increase of angiogenesis;
b.) induction of angiogenesis;
c.) enhanced expression of L1-CAM on endothelial cells;
d.) enhanced expression of L1-CAM on endothelial cells, wherein said expression of L1-CAM correlates with increased vascularization of the tissue affected by the disease;
or a combination thereof.

34. The method of claim 31, wherein the antibody binds to L1-CAM expressed on endothelial cells.

35. The method of claim 31, wherein said disease is selected from the group consisting of a tumorigenic disease, an ocular disease, a chronic inflammatory disease, an inflammatory skin disease, a neuroinflammatory disease, Crohn's disease, bartonellosis, transplanted organ rejection, and a stomach ulcer.

36. The method of claim 35, wherein the effective amount of L1-CAM antibody is in a dosage range of about 20-500 μg/kg body weight for intravenous administration, or in a dosage range of about 0.01 pg/kg to 1 mg/kg body weight for intranasal administration.

37. The method of claim 35, wherein said disease is a tumorigenic disease.

38. The method of claim 35, characterized in that
the ocular disease is selected from the group consisting of macular degeneration such as age related macular degeneration, or retinopathy such as proliferative diabetic retinopathy or retinopathy of prematurity, ocular neovascularization, neovascular glaucoma, corneal neovasculature, retinal vein occlusion, or retinal artery occlusion;
the chronic inflammatory disease is selected from the group consisting of an arthritic disease, more preferably rheumatoid arthritis, osteoarthritis, psoriatic arthritis, spondyloarthropathies, ankylosing spondylitis, Churg-Strauss syndrome, fibromyalgia, giant cell arthritis, gout, Henoch-Schoenlein Purpura, hypersensitivity vasculitis, polyarthritis nodosa, systemic lupus erythematosus, systemic sclerosis, takayasu arthritis, Wegener's granulomatosis or morbus Reiter;
the inflammatory skin disease is dermatitis or psoriasis;
the neuroinflammatory disease is multiple sclerosis; or
the tumorigenic disease is a solid or hematogenous tumor.

39. The method of claim 31, wherein the antibody binds to L1-CAM expressed on endothelial cells, wherein said endothelial cells are tumor endothelial cells.

40. The method of claim 35, wherein said tumorigenic disease is provoked by a tumor selected from the group consisting of astrocytoma, oligodendroglioma, meningioma, neurofibroma, glioblastoma, ependymoma, Schwannoma, neurofibrosarcoma, medulloblastoma, melanoma, preferably malignant melanoma, pancreatic cancer, prostate carcinoma, head and neck cancer, breast cancer, lung cancer, preferably small cancer, non-small cancer, colon cancer, preferably adenocarcinoma of the colon, colorectal cancer, gastrointestinal stromal tumor, ovarian cancer, endometrial cancer, renal cancer, neuroblastomas, squamous cell carcinomas, medulloblastomas, hepatoma and mesothelioma, epidermoid carcinoma, clear cell adenocarcinoma and serous adenocarcinoma of the uterine corps, cervix carcinoma, urinary tract adenocarcinoma, Pheochromocytoma, neuroma, neurillemoma, and paranganglioma, preferably pancreas carcinoma.

41. The method of claim 31, wherein said antibody is used in combination with at least one additional therapeutic agent or in combination with radiotherapy.

42. The method of claim 31, wherein said antibody is used in combination with at least one additional therapeutic agent or in combination with radiotherapy, wherein said additional therapeutic agent is an anti-angiogenic agent.

43. The method of claim 31, wherein said antibody is used in combination with at least one additional therapeutic agent or in combination with radiotherapy, wherein said additional therapeutic agent is an anti-angiogenic agent selected from the group consisting of
a.) agents that target the vascular endothelial growth factor (VEGF) pathway,
b.) an integrin,
c.) a matrix metalloproteinase (MMP),
d.) protein kinase C beta (PKCβ),
e.) a cationic liposome,
f.) a Vascular Targeting Agent (VTA),
g.) Neovastat (AE-941),
h.) U-995,
i.) Squalamine,
j.) Thalidomide or one of its immunomodulatory analogs,
or a combination thereof.

44. The method of claim 31, wherein said antibody is used in combination with at least one additional therapeutic agent or in combination with radiotherapy, wherein said additional therapeutic agent is an anti-angiogenic agent selected from the group consisting of agents that target the vascular endothelial growth factor (VEGF) pathway, an integrin, a matrix metalloproteinase (MMP), protein kinase C beta (PKCβ), or a combination thereof, wherein said anti-angiogenic agent is preferably selected from the group consisting of:
a.) an anti-angiogenic agent targeting an MMP or an integrin that is a chimeric, humanized or fully human monoclonal antibody;
b.) an anti-angiogenic agent targeting an MMP that is selected from the group consisting of marimastat, metastat (COL-3), BAY-129566, CGS-27023, prinomastat (AG-3340), and BMS-27529; or
c.) an anti-angiogenic agent targeting an integrin that is selected from the group consisting of SB-267268, JSM6427, and EMD270179.

45. A method for preventing the formation of a tumor metastasis in a human patient, wherein an effective amount of said anti L1-CAM antibody is administered to said patient.

46. The method of claim 45, wherein the effective amount of L1-CAM antibody is in a dosage range of about 20-500 μg/kg body weight for intravenous administration, or in a dosage range of about 0.01 pg/kg to 1 mg/kg body weight for intranasal administration.

47. The method of claim 45, wherein said prevention is mediated by inhibition of tumor cell adhesion to endothelial cells, tumor cell transmigration over endothelial cells, or a combination thereof.

48. The method of claim 45, wherein said prevention is mediated by inhibition of tumor cell adhesion to endothelial cells, tumor cell transmigration over endothelial cells, or a combination thereof, further characterized in that
said inhibition correlates with enhanced expression of L1-CAM on said endothelial cells;
b.) said endothelial cells are tumor endothelial cells; or
c.) said inhibition correlates with enhanced expression of L1-CAM on said endothelial cells and said endothelial cells are tumor endothelial cells.

49. The method of claim 45, wherein said tumor metastasis is originates from a tumor selected from the group of tumors consisting of astrocytoma, oligodendroglioma, meningioma, neurofibroma, glioblastoma, ependymoma, Schwannoma, neurofibrosarcoma, medulloblastoma, melanoma, preferably malignant melanoma, pancreatic cancer, prostate carcinoma, head and neck cancer, breast cancer, lung cancer, preferably small cancer, non-small cancer, colon cancer, preferably adenocarcinoma of the colon, colorectal cancer, gastrointestinal stromal tumor, ovarian cancer, endometrial cancer, renal cancer, neuroblastomas, squamous cell carcinomas, medulloblastomas, hepatoma and mesothelioma, epidermoid carcinoma, clear cell adenocarcinoma and serous adenocarcinoma of the uterine corps, cervix carcinoma, urinary tract adenocarcinoma, Pheochromocytoma, neuroma, neurillemoma, and paranganglioma, preferably pancreas carcinoma.

50. The method of claim 31, wherein said antibody is a polyclonal or a monoclonal antibody.

51. The method of claim 31, wherein the epitope of said antibody is within the immunoglobulin-like domains of L1 or within the first immunoglobulin-like domain of L1.

52. The method of claim 31, wherein the epitope of said antibody is within the first immunoglobulin-like domain of L1 and wherein said antibody is characterized in that
a.) the antibody is capable of binding to the same L1 epitope recognized by the monoclonal antibody 9.3. produced by the hybridoma cell deposited under DSMZ ACC2841;
b.) said antibody has the same capacity to inhibit tumor growth as the monoclonal antibody 9.3. produced by the hybridoma cell deposited under DSMZ ACC2841;
c.) said antibody is a monoclonal antibody produced by the hybridoma cell deposited under DSMZ ACC2841;
d.) at least one of its complementarity determining regions (CDRs)
i.) has one of the following sequences RASQDISNYLN (SEQ ID NO: 1), YTSRLHS (SEQ ID NO: 2), QQGNTLPWT (SEQ ID NO: 3), RYWML (SEQ ID NO: 4), EINPRNDRTNYNEKFKT (SEQ ID NO: 5), or GGGYAMDY (SEQ ID NO: 6) or
ii.) has a sequence which, in comparison to the sequences mentioned under 1) has at least one conservative amino acid exchange.

53. The method of claim 31, wherein said antibody is a humanized antibody based on an antibody characterized in that
said antibody is a polyclonal or a monoclonal antibody;
the epitope of said antibody is within the immunoglobulin-like domains of L1;
the epitope of said antibody is within the first immunoglobulin-like domain of L1;
said antibody is capable of binding to the same L1 epitope recognized by the monoclonal antibody 9.3. produced by the hybridoma cell deposited under DSMZ ACC2841;
said antibody has the same capacity to inhibit tumor growth as the monoclonal antibody 9.3. produced by the hybridoma cell deposited under DSMZ ACC2841;
said antibody is a monoclonal antibody that is produced by the hybridoma cell deposited under DSMZ ACC2841;
said antibody is characterized in that at least one of its complementarity determining regions (CDRs) having
i.) one of the following sequences RASQDISNYLN (SEQ ID NO: 1), YTSRLHS (SEQ ID NO: 2), QQGNTLPWT (SEQ ID NO: 3), RYWML (SEQ ID NO: 4), EINPRNDRTNYNEKFKT (SEQ ID NO: 5), or GGGYAMDY (SEQ ID NO: 6) or
ii.) a sequence which, in comparison to the sequences mentioned under i) has at least one conservative amino acid exchange; or
said antibody is a humanized antibody having at least one non-human CDR and human framework region (FR) residues.

54. The method of claim 45, wherein said antibody is a polyclonal or a monoclonal antibody.

55. The method of claim 45, wherein the epitope of said antibody is within the immunoglobulin-like domains of L1 or within the first immunoglobulin-like domain of L1.

56. The method of claim 45, wherein the epitope of said antibody is within the first immunoglobulin-like domain of L1 and wherein said antibody is characterized in that
a.) said antibody is capable of binding to the same L1 epitope recognized by the monoclonal antibody 9.3. produced by the hybridoma cell deposited under DSMZ ACC2841;
b.) said antibody has the same capacity to inhibit tumor growth as the monoclonal antibody 9.3. produced by the hybridoma cell deposited under DSMZ ACC2841;
c.) said antibody is a monoclonal antibody produced by the hybridoma cell deposited under DSMZ ACC2841;
d.) at least one of its complementarity determining regions (CDRs)
i.) has one of the following sequences RASQDISNYLN (SEQ ID NO: 1), YTSRLHS (SEQ ID NO: 2), QQGNTLPWT (SEQ ID NO: 3), RYWML (SEQ ID NO: 4), EINPRNDRTNYNEKFKT (SEQ ID NO: 5), or GGGYAMDY (SEQ ID NO: 6) or
ii.) has a sequence which, in comparison to the sequences mentioned under i) has at least one conservative amino acid exchange.

57. The method of claim 45, wherein said antibody is a humanized antibody based on an antibody characterized in that
said antibody is a polyclonal or a monoclonal antibody;
the epitope of said antibody is within the immunoglobulin-like domains of L1;
the epitope of said antibody is within the first immunoglobulin-like domain of L1;
said antibody is capable of binding to the same L1 epitope recognized by the monoclonal antibody 9.3. produced by the hybridoma cell deposited under DSMZ ACC2841;
said antibody has the same capacity to inhibit tumor growth as the monoclonal antibody 9.3. produced by the hybridoma cell deposited under DSMZ ACC2841;
said antibody is a monoclonal antibody that is produced by the hybridoma cell deposited under DSMZ ACC2841;
said antibody is characterized in that at least one of its complementarity determining regions (CDRs) having
i.) one of the following sequences RASQDISNYLN (SEQ ID NO: 1), YTSRLHS (SEQ ID NO: 2), QQGNTLPWT (SEQ ID NO: 3), RYWML (SEQ ID NO: 4), EINPRNDRTNYNEKFKT (SEQ ID NO: 5), or GGGYAMDY (SEQ ID NO: 6) or
ii.) a sequence which, in comparison to the sequences mentioned under i) has at least one conservative amino acid exchange; or
said antibody is a humanized antibody having at least one non-human CDR and human framework region (FR) residues.


SEQUENCE LISTING (6 sequences)

SEQ ID NO: 1 - protein, 11 aa, Artificial Sequence
  Description of Artificial Sequence Synthetic CDR sequence of the monoclonal antibody 9.3 determined according to the method of Kabat
RASQDISNYLN

SEQ ID NO: 2 - protein, 7 aa, Artificial Sequence
  Description of Artificial Sequence Synthetic CDR sequence of the monoclonal antibody 9.3 determined according to the method of Kabat
YTSRLHS

SEQ ID NO: 3 - protein, 9 aa, Artificial Sequence
  Description of Artificial Sequence Synthetic CDR sequence of the monoclonal antibody 9.3 determined according to the method of Kabat
QQGNTLPWT

SEQ ID NO: 4 - protein, 5 aa, Artificial Sequence
  Description of Artificial Sequence Synthetic CDR sequence of the monoclonal antibody 9.3 determined according to the method of Kabat
RYWML

SEQ ID NO: 5 - protein, 17 aa, Artificial Sequence
  Description of Artificial Sequence Synthetic CDR sequence of the monoclonal antibody 9.3 determined according to the method of Kabat
EINPRNDRTNYNEKFKT

SEQ ID NO: 6 - protein, 8 aa, Artificial Sequence
  Description of Artificial Sequence Synthetic CDR sequence of the monoclonal antibody 9.3 determined according to the method of Kabat
GGGYAMDY